(19) United States
(12) Patent Application Publication
(10) Pub. No.: US 2024/0309385 A1
(43) Pub. Date: Sep. 19, 2024

(54) COMPOSITIONS AND METHODS RELATED TO NUCLEIC ACID ANTICOAGULANTS AND ANTIDOTES

(71) Applicant: North Carolina State University, Raleigh, NC (US)

(72) Inventors: Abhichart Krissanaprasit, Raleigh, NC (US); Thom LaBean, Raleigh, NC (US)

(21) Appl. No.: 18/681,684

(22) PCT Filed: Aug. 5, 2022
(86) PCT No.: PCT/US2022/074602

§ 371 Date: Feb. 6, 2024

Related U.S. Application Data
(60) Provisional application No. 63/230,153, filed on Aug. 6, 2021.

(51) Int. Cl. C12N 15/113; A61P 7/02; C12N 15/115

(52) U.S. Cl. CPC C12N 15/1137; A61P 7/02; C12N 15/115; C12N 2310/16

(57) ABSTRACT

The present disclosure provides compositions and methods related to nucleic acid molecules having therapeutic activity. In particular, the present disclosure provides nucleic acid molecules comprising one or more aptamers having anticoagulant activity, as well as corresponding antidote nucleic acid molecules capable of modulating anticoagulant activity.

### CROSS REFERENCE TO RELATED APPLICATIONS

This application claims priority to and the benefit of U.S. Provisional Patent Application No. 63/230,153 filed Aug. 6, 2021, which is incorporated herein by reference in its entirety for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: 35,590 Byte (XML) file named “NCSU-39417-601” created on Aug. 4, 2022.

### FIELD

The present disclosure provides compositions and methods related to nucleic acid molecules having therapeutic activity. In particular, the present disclosure provides nucleic acid molecules comprising one or more aptamers having anticoagulant activity, as well as corresponding antidote nucleic acid molecules capable of modulating anticoagulant activity.

### BACKGROUND

The coagulation cascade involves a series of enzymatic reactions that ultimately produce fibrin clots on ruptured vascular and cellular surfaces. Anticoagulants disrupt the process of coagulation by blocking key players in the cascade. The regulation of fibrin clot formation by anticoagulants can consequently evade thrombosis, the formation of blood clots, in vital organs such as the heart, lungs, and brain. The life-threatening ramifications of thrombosis include strokes or transient ischemic attacks, heart attacks, deep vein thrombosis, and pulmonary embolisms.

The most commonly prescribed anticoagulant is Warfarin, which is a small molecule often used as a rodenticide. Warfarin is a vitamin K antagonist that inhibits the synthesis of clotting factors II, VII, IX, and X and endogenous anticoagulant proteins C and S. The body's sensitivity to vitamin K fluctuations requires strict and timely monitoring of its levels and adjusts dosages accordingly. Other forms of anticoagulants include Heparins, Factor Xa Inhibitors, Direct Thrombin Inhibitors, and Fibrobrinolytics. Consistent across all current methods of anticoagulation is a narrow therapeutic window for administration that effectively treats clotting without causing excessive anticoagulation, for example, during surgery if dosage of an anticoagulant is too high. Unfortunately, there is no antidote for chemical-based anticoagulants that can further mediate administration and combat the cytotoxic effects.

### SUMMARY

Embodiments of the present disclosure include a single-stranded nucleic acid molecule comprising at least one RNA aptamer reversal element that is complementary to at least a portion of an RNA aptamer having anti-coagulation activity.

In some embodiments, binding of the at least one RNA aptamer reversal element to the at least one RNA aptamer having anti-coagulation activity counteracts the anti-coagulation activity.

In some embodiments, binding of the at least one RNA aptamer reversal element to the at least one RNA aptamer having anti-coagulant activity counteracts the anti-coagulation activity by at least about 25%. In some embodiments, binding of the at least one RNA aptamer reversal element to the at least one RNA aptamer having anti-coagulant activity counteracts the anti-coagulation activity by at least about 50%. In some embodiments, binding of the at least one RNA aptamer reversal element to the at least one RNA aptamer having anti-coagulant activity counteracts the anti-coagulation activity by at least about 75%.

In some embodiments, the at least one RNA aptamer reversal element binds to the at least one RNA aptamer with at least about 50% complementarity. In some embodiments, at least one RNA aptamer reversal element binds to the at least one RNA aptamer with at least about 75% complementarity. In some embodiments, the at least one RNA aptamer reversal element binds to the at least one RNA aptamer with at least about 95% complementarity.

In some embodiments, the nucleic acid comprises at least two RNA aptamer reversal elements that are complementary to at least a portion of a single RNA aptamer having anti-coagulation activity. In some embodiments, the nucleic acid comprises at least two RNA aptamer reversal elements that are complementary to at least a portion of two different RNA aptamers having anti-coagulation activity.

In some embodiments, the at least two RNA aptamer reversal elements are separated by a linker region. In some embodiments, the linker region comprises from about 1 to about 50 nucleotides. In some embodiments, the at least two RNA aptamer reversal elements are continuous and not separated by a linker region.

In some embodiments, the anti-coagulation activity of the RNA aptamer comprises inhibition of one or more of Factor XIIa, Factor XIIIa, Factor XIa, Factor IXa, Factor Xa, and/or von Willebrand factor.

In some embodiments, the anti-coagulation activity of the RNA aptamer comprises thrombin inhibition. In some embodiments, the RNA aptamer is capable of binding exosite 1 of thrombin. In some embodiments, the RNA aptamer is capable of binding exosite 2 of thrombin. In some embodiments, the RNA aptamer is an anti-thrombin RNA_{R9D-14T} aptamer or a derivative thereof. In some embodiments, the RNA aptamer comprises an anti-thrombin Toggle-25t RNA aptamer or a derivative thereof.

In some embodiments, the at least one RNA aptamer reversal element comprises a sequence that is at least 80% identical to SEQ ID NOs: 1 or 2. In some embodiments, the nucleic acid comprises a sequence that is at least 80% identical to any of SEQ ID NOs: 3-10.

Embodiments of the present disclosure also include a vector comprising any of the nucleic acid molecule described herein.

Embodiments of the present disclosure also include a therapeutic composition comprising any of the nucleic acid molecules described herein and a pharmaceutically acceptable excipient, solvent, carrier, or diluent.

Embodiments of the present disclosure also include a method of modulating coagulation in a subject in need thereof. In accordance with these embodiments, the method includes administering any of the therapeutic compositions described herein to a subject.

In some embodiments, the administration of a therapeutic compositions described herein to a subject is performed after the subject has been administered a composition comprising an RNA aptamer having anti-coagulation activity.

Embodiments of the present disclosure also include a system or kit for modulating coagulation. In accordance with these embodiments, the system includes any of the nucleic acid molecules described herein and an RNA aptamer having anti-coagulation activity.

### BRIEF DESCRIPTION OF THE DRAWINGS

FIGS. 1A-1H: Representative 2D illustration of 2HF-2211 RNA origami anticoagulant and specific, single-stranded DNA antidote (Anti-HEX21) (FIG. 1A). Characterization of 2HF-2211 using denaturing polyacrylamide gel electrophoresis (FIG. 1B). Anticoagulation activity of 2HF-2211 using activated partial thromboplastin time (aPTT) assay in human, porcine and murine plasma (FIGS. 1C-1E). Anticoagulation activity of 2HF-2211 using prothrombin time (PT) assay in human, porcine and murine plasma (FIGS. 1F-1H).

FIGS. 2A-2B: Comparison of nine reversal agents (i.e., antidotes) when combined with anticoagulant 2HF-2211 in aPTT assay. RNA origami anticoagulant reversal by various antidotes (anticoagulant:antidote ratio is 1:5) (FIG. 2A). Dose response of single-stranded DNA antidote Anti-HEX21_0nt (no spacer) used as reversal agent (FIG. 2B).

FIG. 3: Time course of re-establishment of coagulation following reversal of 2HF-2211 anticoagulant activity by AntiHex21_0nt antidote. RNA origami:antidote ratio is 1:5.

FIGS. 4A-4B: In vivo efficacy of 2HF-2211 RNA anticoagulant in a whole mouse liver laceration model. Increased total blood loss (FIG. 4A) and cumulative blood loss (FIG. 4B) demonstrate RNA anticoagulation activity at 1 and 3 mg/kg) equal to a high dose of heparin (500 U/kg).

FIGS. 5A-5B: In vivo reversal of 2HF-2211 RNA anticoagulant activity by antidote Anti-HEX21_0nt in a whole mouse liver laceration model. Increased total blood loss (FIG. 5A) and cumulative blood loss (FIG. 5B) are shown.

FIG. 6: Secondary structure and complementary binding predictions by NUPACK computational modeling for antidote Anti-HEX21_0nt and in combination with exosite-1 and -2 aptamers.

FIG. 7: Secondary structure and complementary binding predictions by NUPACK computational modeling for antidote Anti-HEX12_31nt and in combination with exosite-1 and -2 aptamers.

FIG. 8: Secondary structure and complementary binding predictions by NUPACK computational modeling for antidote Anti-HEX21_11nt linker and in combination with exosite-1 and -2 aptamers.

FIG. 9: Secondary structure and complementary binding predictions by NUPACK computational modeling for antidote Anti-HEX21_21nt linker and in combination with exosite-1 and -2 aptamers.

FIG. 10: Secondary structure and complementary binding predictions by NUPACK computational modeling for antidote Anti-HEX21_31nt linker and in combination with exosite-1 and -2 aptamers.

FIG. 11: Secondary structure and complementary binding predictions by NUPACK computational modeling for antidote Anti-HEX12_0nt and in combination with exosite-1 and -2 aptamers.

FIG. 12: Secondary structure and complementary binding predictions by NUPACK computational modeling for antidote Anti-HEX12_11nt and in combination with exosite-1 and -2 aptamers.

FIG. 13: Secondary structure and complementary binding predictions by NUPACK computational modeling for antidote Anti-HEX12_21nt and in combination with exosite-1 and -2 aptamers.

FIGS. 14A-14I: Representative 2D models of the RNA origami described in the present disclosure.

### DETAILED DESCRIPTION

Embodiments of the present disclosure provide RNA aptamer antidotes or reversal agents that modulate the anticoagulation activity of an RNA-based aptamer. For example, an RNA aptamer exhibiting anticoagulation activity via direct thrombin inhibition known as 2HF-2211 is described in International PCT Appln. PCT/US19/58133, filed Oct. 25, 2019, which claims priority to U.S. Provisional Application No. 62/750,900, filed Oct. 26, 2018, both of which are incorporated herein by reference in their entireties. This exemplary RNA aptamer exhibiting anticoagulation activity is further described in A. Krissanaprasit, et al., (2021) Multivalent Aptamer-Functionalized Single-Strand RNA Origami as Effective, Target-Specific Anticoagulants with Corresponding Reversal Agents, Advanced Healthcare Materials. 2021, 2001826 (DOI: 10.1002/adhm.202001826).

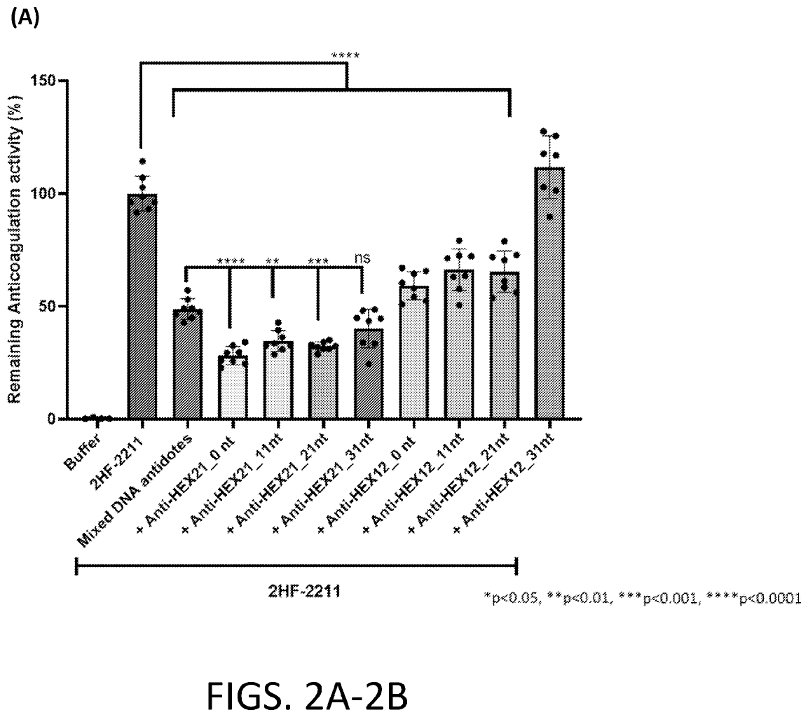

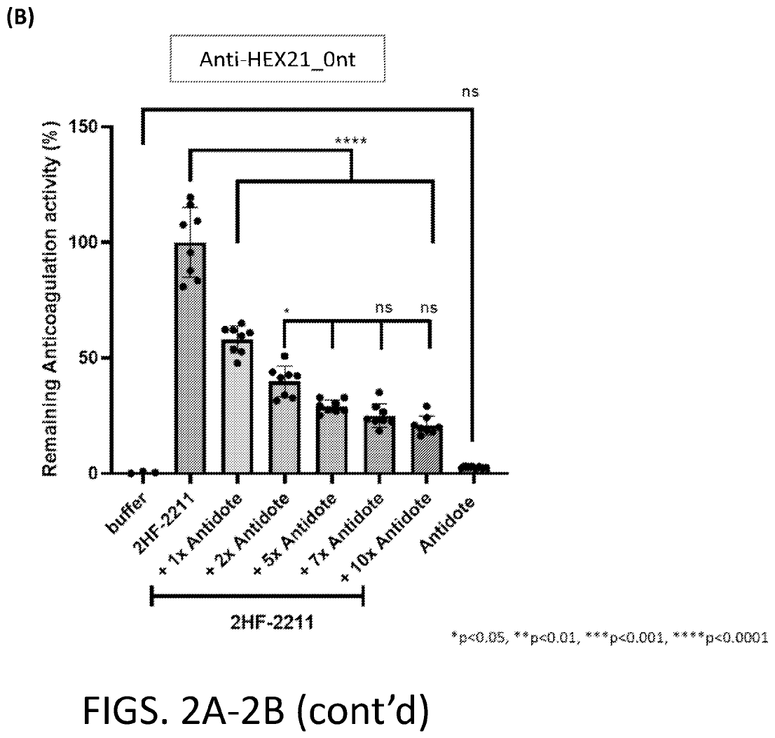

As described further herein, the RNA aptamer antidotes or reversal agents of the present disclosure bind via Watson-Crick complementarity to thrombin-binding aptamers incorporated within a single anticoagulant molecule (e.g., 2HF-2211). Embodiments of the present disclosure provide single-molecule DNA agents engineered to contain aptamer reversal-agent/antidotes fused directly with no spacer (0nt) and/or linked by DNA spacers of various nucleotide lengths. Additionally, the other molecular design choice explored and optimized involved the order of the reversal sequences along the 5′-to-3′ direction of the antidote DNA strand. For example, in one embodiment, either an Exosite 1 reversal sequence was placed before an Exosite 2 reversal sequence (Anti-HEX12), or an Exosite 2 reversal sequence was placed before an Exosite 1 reversal sequence (Anti-HEX21). As described further below, one reversal agent tested was Anti-HEX21-0nt (see, e.g., FIG. 2), with no linker sequence (zero nucleotides) between the two reversal sequences, and the Exosite 2 reversal sequence placed before the Exosite 1 reversal sequence in the 5′-to-3′ direction of the single molecule, bifunctional, DNA antidote strand. In another embodiment, one of the designs tested (i.e., Anti-HEX12-31nt), with the opposite ordering of the reversal sequences and the longest tested linker/spacer sequence (31 nucleotides), actually increased (rather than inhibited) the activity of the anticoagulant (see, e.g., FIG. 2).

As would be recognized by one of ordinary skill in the art based on the present disclosure, the various methods and compositions described herein pertaining to the design and optimization of nucleic acid molecules comprising an RNA aptamer reversal element(s) can be applied to any complementary RNA aptamer, including but not limited to, RNA aptamers having anticoagulant activity.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The term “aptamer” generally refers to either an oligonucleotide of a single defined sequence or a mixture of said oligonucleotides, wherein the mixture retains the properties of binding specifically to a target molecule. Thus, as used herein “aptamer” denotes both singular and plural sequences of oligonucleotides. The term “aptamer” generally refers to a single-stranded or double-stranded nucleic acid which is capable of binding to a protein or other molecule, and thereby disturbing the protein's or other molecule's function.

The term “single-stranded” oligonucleotides generally refers to those oligonucleotides that contain a single covalently linked series of nucleotide residues.

The terms “oligomers” or “oligonucleotides” include RNA or DNA sequences of more than one nucleotide in either single chain or duplex form and specifically includes short sequences such as dimers and trimers, in either single chain or duplex form, which can be intermediates in the production of the specifically binding oligonucleotides. “Modified” forms used in candidate pools contain at least one non-native residue. “Oligonucleotide” or “oligomer” is generic to polydeoxyribonucleotides (containing 2′-deoxy-D-ribose or modified forms thereof), such as DNA, to polyribonucleotides (containing D-ribose or modified forms thereof), such as RNA, and to any other type of polynucleotide which is an N-glycoside or C-glycoside of a purine or pyrimidine base, or modified purine or pyrimidine base or abasic nucleotides. Oligonucleotide” or “oligomer” can also be used to describe artificially synthesized polymers that are similar to RNA and DNA, including, but not limited to, oligos of peptide nucleic acids (PNA).

An “RNA aptamer” is an aptamer comprising ribonucleoside units. “RNA aptamer” is also meant to encompass RNA analogs as disclosed herein.

The term “coagulation factor” generally refers to a factor that acts in either or both of the intrinsic and the extrinsic coagulation cascade.

The term “RNA analog” or “RNA derivative” or “modified RNA” generally refer to a polymeric molecule, which in addition to containing ribonucleosides as its units, also contains at least one of the following: 2′-deoxy, 2′-halo (including 2′-fluoro), 2′-amino (preferably not substituted or mono- or disubstituted), 2′-mono-, di-or tri-halomethyl, 2′-O-alkyl, 2′-O-halo-substituted alkyl, 2′-alkyl, azido, phosphorothioate, sulfhydryl, methylphosphonate, fluorescein, rhodamine, pyrene, biotin, xanthine, hypoxanthine, 2,6-diamino purine, 2-hydroxy-6-mercaptopurine and pyrimidine bases substituted at the 6-position with sulfur or 5 position with halo or C₁₋₅ alkyl groups, a basic linkers, 3′-deoxy-adenosine as well as other available “chain terminator” or “non-extendible” analogs (at the 3′-end of the RNA), or labels such as ³³P, ³³P and the like. All of the foregoing can be incorporated into an RNA using the standard synthesis techniques disclosed herein.

The terms “binding activity” and “binding affinity” generally refer to the tendency of a ligand molecule to bind or not to bind to a target. The energetics of these interactions are significant in “binding activity” and “binding affinity” because they can include definitions of the concentrations of interacting partners, the rates at which these partners are capable of associating, and the relative concentrations of bound and free molecules in a solution.

“Sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

### 2. COMPOSITIONS AND SYSTEMS

Functional RNA origami. Embodiments of the present disclosure provide single-stranded nucleic acid molecules having an A-form double-helical structure and at least one crossover region, at least one kissing-loop region, and at least one nucleic acid aptamer having anti-coagulant activity. In accordance with these embodiments, the single-stranded nucleic acid molecule can be a DNA molecule or an RNA molecule, or any derivatives or combinations thereof.

In some embodiments, the single-stranded nucleic acid molecule can be an RNA molecule that includes at least one nucleoside with a 2′-modification. In some embodiments, single-stranded RNA origami molecules of the present disclosure can include at least one 2′-fluoro-dCTP or a 2′-fluoro-dUTP or one other nucleoside with a 2′-modification such as 2′-amino or 2′-O-methyl or a chemical modification of the backbone phosphate groups such as a phosphorothioate.

In some embodiments, the single-stranded nucleic acid molecule includes at least one tetra-loop region comprising a four-nucleotide motif. In some embodiments, the single-stranded nucleic acid molecule includes from one to three tetra-loop regions, each comprising a four-nucleotide motif. In some embodiments, the single-stranded nucleic acid molecule includes from one to four aptamers having anti-coagulant activity. In accordance with these embodiments, each of the from one to four aptamers replaces one of the at least one tetra-loop regions. In some embodiments, the single-stranded nucleic acid molecule does not include a tetra-loop region.

In some embodiments, the single-stranded nucleic acid molecule includes at least one kissing-loop region is a 180° kissing loop region. In some embodiments, the single-stranded nucleic acid molecule includes one 180° kissing loop region. In some embodiments, the single-stranded nucleic acid molecule does not include a kissing loop region.

In some embodiments, the single-stranded nucleic acid molecule includes single-stranded RNA linker region. In some embodiments, nucleic acid aptamers can be linked to one end or both ends of the single stranded RNA linker region. In some embodiments, the single-stranded nucleic acid molecule does not include a kissing loop region or a tetraloop region.

In some embodiments, the single-stranded nucleic acid molecule includes at least one helical structure (e.g., an A-form double-helical structure). In some embodiments, the single-stranded nucleic acid molecule includes at least two helical structures. In some embodiments, the single-stranded nucleic acid molecule includes at least three helical structures. In some embodiments, the single-stranded nucleic acid molecule includes at least four helical structures. In some embodiments, the single-stranded nucleic acid molecule includes five or more helical structures. In some embodiments, the single-stranded nucleic acid molecule includes at least one helical structure separating two or more nucleic acid aptamers. In some embodiments, the at least one helical structure separating two or more nucleic acid aptamers includes at least one nucleic acid aptamer. In some embodiments, the at least one helical structure separating two or more nucleic acid aptamers does not include a nucleic acid aptamer.

In some embodiments, the nucleic acid molecule is an RNA molecule having at least 80% sequence identity to any of SEQ ID NOs: 11-29. In some embodiments, the nucleic acid molecule is an RNA molecule having at least 85% sequence identity to any of SEQ ID NOS: 11-29. In some embodiments, the nucleic acid molecule is an RNA molecule having at least 90% sequence identity to any of SEQ ID NOs: 11-29. In some embodiments, the nucleic acid molecule is an RNA molecule having at least 95% sequence identity to any of SEQ ID NOs: 11-29. In some embodiments, the nucleic acid molecule is an RNA molecule having at least 96% sequence identity to any of SEQ ID NOs: 11-29. In some embodiments, the nucleic acid molecule is an RNA molecule having at least 97% sequence identity to any of SEQ ID NOS: 11-29. In some embodiments, the nucleic acid molecule is an RNA molecule having at least 98% sequence identity to any of SEQ ID NOs: 11-29. In some embodiments, the nucleic acid molecule is an RNA molecule having at least 99% sequence identity to any of SEQ ID NOs: 11-29.

In some embodiments, the anti-coagulation activity of the at least one nucleic acid aptamer includes the inhibition of one or more of Factor XIIa, Factor XIIIa, Factor XIa, Factor IXa, Factor Xa, and von Willebrand factor. In some embodiments, the nucleic acid aptamers that can be included in the RNA origami molecules disclosed herein include any aptamers involved in modulating blood coagulation, including but not limited to, ARC183/HD1 (targets FIIa); HD22 (targets FIIa); HD1-22 (targets FIIa); Tog25 (targets FII); R9d14t (targets FII/FIIa); 11F7t (targets FXa); 16.3 (targets FVIIa); 7S-1/7S-2 (targets FVII); 9.3t (targets FIXa); R4cXII-1 (targets FXII/FXIIa); NU172 (targets thrombin); REG1 (targets FIX/FIXa); REG2 (targets FIX/FIXa); ARC1779 (targets von Willebrand Factor); and ARC19499 (targets TFPI).

In some embodiments, the anti-coagulation activity of the at least one nucleic acid aptamer comprises thrombin inhibition. In some embodiments, the at least one nucleic acid aptamer comprises an anti-thrombin RNA_{R9D-14T} aptamer or a derivative thereof. In some embodiments, the at least one nucleic acid aptamer comprises an anti-thrombin Toggle-25t RNA aptamer or a derivative thereof. In some embodiments, the nucleic acid aptamer capable of binding exosite 1 of thrombin is an RNA_{R9D-14T} aptamer or a derivative thereof. In some embodiments, the nucleic acid aptamer capable of binding exosite 2 of thrombin is a Toggle-25t RNA aptamer or a derivative thereof.

In some embodiments, the nucleic acid molecule is an RNA molecule comprising from about 100 to about 1000 nucleotides. In some embodiments, the nucleic acid molecule is an RNA molecule comprising from about 100 to about 900 nucleotides. In some embodiments, the nucleic acid molecule is an RNA molecule comprising from about 100 to about 800 nucleotides. In some embodiments, the nucleic acid molecule is an RNA molecule comprising from about 100 to about 700 nucleotides. In some embodiments, the nucleic acid molecule is an RNA molecule comprising from about 100 to about 600 nucleotides. In some embodiments, the nucleic acid molecule is an RNA molecule comprising from about 100 to about 500 nucleotides. In some embodiments, the nucleic acid molecule is an RNA molecule comprising from about 100 to about 400 nucleotides. In some embodiments, the nucleic acid molecule is an RNA molecule comprising from about 100 to about 300 nucleotides. In some embodiments, the nucleic acid molecule is an RNA molecule comprising from about 150 to about 600 nucleotides. In some embodiments, the nucleic acid molecule is an RNA molecule comprising from about 150 to about 500 nucleotides. In some embodiments, the nucleic acid molecule is an RNA molecule comprising from about 150 to about 400 nucleotides. In some embodiments, the nucleic acid molecule is an RNA molecule comprising from about 150 to about 300 nucleotides. In some embodiments, the nucleic acid molecule is an RNA molecule comprising from about 200 to about 600 nucleotides. In some embodiments, the nucleic acid molecule is an RNA molecule comprising from about 225 to about 600 nucleotides. In some embodiments, the nucleic acid molecule is an RNA molecule comprising from about 250 to about 600 nucleotides. In some embodiments, the nucleic acid molecule is an RNA molecule comprising from about 200 to about 500 nucleotides. In some embodiments, the nucleic acid molecule is an RNA molecule comprising from about 200 to about 450 nucleotides. In some embodiments, the nucleic acid molecule is an RNA molecule comprising from about 200 to about 400 nucleotides. In some embodiments, the nucleic acid molecule is an RNA molecule comprising from about 200 to about 350 nucleotides.

In some embodiments, the single-stranded nucleic acid molecule is an RNA molecule that comprises a nucleic acid aptamer that replaces tetra-loop region 1 of the RNA molecule capable of binding exosite 1 of thrombin, and a nucleic acid aptamer that replaces tetra-loop region 2 of the RNA molecule capable of binding exosite 2 of thrombin (2HO-RNA-12NN or 2HF-RNA-12NN). In some embodiments, the single-stranded nucleic acid molecule is an RNA molecule that comprises a nucleic acid aptamer that replaces tetra-loop region 1 of the RNA molecule capable of binding exosite 1 of thrombin, and a nucleic acid aptamer that replaces tetra-loop region 3 of the RNA molecule capable of binding exosite 2 of thrombin (2HO-RNA-IN2N or 2HF-RNA-IN2N). In some embodiments, the single-stranded nucleic acid molecule is an RNA molecule that comprises a nucleic acid aptamer that replaces tetra-loop region 1 of the RNA molecule capable of binding exosite 2 of thrombin, and a nucleic acid aptamer that replaces tetra-loop region 4 of the RNA molecule capable of binding exosite 1 of thrombin (2HO-RNA-2NN1 or 2HF-RNA-2NN1). In some embodiments, the single-stranded nucleic acid molecule is an RNA molecule that comprises a nucleic acid aptamer capable of binding exosite 2 of thrombin linked to one end of a single-stranded RNA linker, and a nucleic acid aptamer capable of binding exosite 1 of thrombin linked to the other end of the single-stranded RNA linker (Fss12). In some embodiments, the single-stranded nucleic acid molecule is an RNA molecule that comprises two nucleic acid aptamers capable of binding exosite 2 of thrombin, each replacing tetra-loop regions 1 and 2 of the RNA molecule, respectively, and two nucleic acid aptamers capable of binding exosite 1 of thrombin, each replacing tetra-loop regions 3 and 4 of the RNA molecule, respectively (2H-2211). In some embodiments, the single-stranded nucleic acid molecule is an RNA molecule that comprises a nucleic acid aptamer capable of binding exosite 2 of thrombin that replaces tetra-loop region 1 of the RNA molecule, a nucleic acid aptamer capable of binding exosite 1 of thrombin that replaces tetra-loop region 4 of the RNA molecule, and an A-form double-helical structure separating the nucleic acid aptamer capable of binding exosite 2 of thrombin from the nucleic acid aptamer capable of binding exosite 1 of thrombin (3H-2NN1). In some embodiments, the single-stranded nucleic acid molecule is an RNA molecule that comprises a nucleic acid aptamer capable of binding exosite 2 of thrombin that replaces tetra-loop region 1 of the RNA molecule, a nucleic acid aptamer capable of binding exosite 1 of thrombin that replaces tetra-loop region 4 of the RNA molecule, and two A-form double-helical structures separating the nucleic acid aptamer capable of binding exosite 2 of thrombin from the nucleic acid aptamer capable of binding exosite 1 of thrombin (4H-2NN1).

Embodiments of the present disclosure also include a DNA molecule encoding any of the single-stranded nucleic acid molecules described herein. As would be recognized by one of ordinary skill in the art based on the present disclosure, the DNA molecule encoding any of the single-stranded nucleic acid molecules described herein can be single- or double-stranded, and can act as a template for generating any of the single-stranded nucleic acid molecules described herein. The DNA template can be part of an expression plasmid or other construct for in vivo and/or in vitro biochemical reactions.

Embodiments of the present disclosure also include an anticoagulant composition. In accordance with these embodiments, the compositions include a single-stranded nucleic acid molecule comprising an A-form double-helical structure and at least one crossover region, at least one kissing-loop region, and at least one nucleic acid aptamer having anti-coagulant activity, and pharmaceutically acceptable excipient, solvent, carrier, or diluent. In some embodiments, the single-stranded nucleic acid molecule additionally comprises at least one tetraloop region. As would be recognized by one of ordinary skill in the art based on the present disclosure, the composition can be administered to a subject or patient in accordance with a treatment regimen to modulate blood coagulation in the context of a surgical procedure and/or to treat a disease condition.

RNA aptamer reversal agents. In accordance with the above embodiments, the present disclosure provides RNA aptamer antidotes or reversal agents that modulate the activity of an RNA-based aptamer. As would be recognized by one of ordinary skill in the art based on the present disclosure, the various methods and compositions described herein pertaining to the design and optimization of nucleic acid molecules comprising an RNA aptamer reversal element(s) can be applied to any complementary RNA aptamer, including but not limited to, RNA aptamers having anticoagulant activity.

In some embodiments, the present disclosure provides a single-stranded nucleic acid molecule comprising at least one RNA aptamer reversal element that is complementary to at least a portion of an RNA aptamer having anti-coagulation activity. In accordance with these embodiments, binding of the at least one RNA aptamer reversal element to the at least one RNA aptamer having anti-coagulation activity counteracts the anti-coagulation activity. In some embodiments, binding of the at least one RNA aptamer reversal element to the at least one RNA aptamer having anti-coagulant activity counteracts the anti-coagulation activity by at least about 10%. In some embodiments, binding of the at least one RNA aptamer reversal element to the at least one RNA aptamer having anti-coagulant activity counteracts the anti-coagulation activity by at least about 15%. In some embodiments, binding of the at least one RNA aptamer reversal element to the at least one RNA aptamer having anti-coagulant activity counteracts the anti-coagulation activity by at least about 20%. In some embodiments, binding of the at least one RNA aptamer reversal element to the at least one RNA aptamer having anti-coagulant activity counteracts the anti-coagulation activity by at least about 25%. In some embodiments, binding of the at least one RNA aptamer reversal element to the at least one RNA aptamer having anti-coagulant activity counteracts the anti-coagulation activity by at least about 30%. In some embodiments, binding of the at least one RNA aptamer reversal element to the at least one RNA aptamer having anti-coagulant activity counteracts the anti-coagulation activity by at least about 35%. In some embodiments, binding of the at least one RNA aptamer reversal element to the at least one RNA aptamer having anti-coagulant activity counteracts the anti-coagulation activity by at least about 40%. In some embodiments, binding of the at least one RNA aptamer reversal element to the at least one RNA aptamer having anti-coagulant activity counteracts the anti-coagulation activity by at least about 45%. In some embodiments, binding of the at least one RNA aptamer reversal element to the at least one RNA aptamer having anti-coagulant activity counteracts the anti-coagulation activity by at least about 50%. In some embodiments, binding of the at least one RNA aptamer reversal element to the at least one RNA aptamer having anti-coagulant activity counteracts the anti-coagulation activity by at least about 55%. In some embodiments, binding of the at least one RNA aptamer reversal element to the at least one RNA aptamer having anti-coagulant activity counteracts the anti-coagulation activity by at least about 60%. In some embodiments, binding of the at least one RNA aptamer reversal element to the at least one RNA aptamer having anti-coagulant activity counteracts the anti-coagulation activity by at least about 65%. In some embodiments, binding of the at least one RNA aptamer reversal element to the at least one RNA aptamer having anti-coagulant activity counteracts the anti-coagulation activity by at least about 70%. In some embodiments, binding of the at least one RNA aptamer reversal element to the at least one RNA aptamer having anti-coagulant activity counteracts the anti-coagulation activity by at least about 75%. In some embodiments, binding of the at least one RNA aptamer reversal element to the at least one RNA aptamer having anti-coagulant activity counteracts the anti-coagulation activity by at least about 80%. In some embodiments, binding of the at least one RNA aptamer reversal element to the at least one RNA aptamer having anti-coagulant activity counteracts the anti-coagulation activity by at least about 85%. In some embodiments, binding of the at least one RNA aptamer reversal element to the at least one RNA aptamer having anti-coagulant activity counteracts the anti-coagulation activity by at least about 90%. In some embodiments, binding of the at least one RNA aptamer reversal element to the at least one RNA aptamer having anti-coagulant activity counteracts the anti-coagulation activity by at least about 95%.

In some embodiments, the at least one RNA aptamer reversal element binds to the at least one RNA aptamer with at least about 25% complementarity. In some embodiments, at least one RNA aptamer reversal element binds to the at least one RNA aptamer with at least about 30% complementarity. In some embodiments, the at least one RNA aptamer reversal element binds to the at least one RNA aptamer with at least about 35% complementarity. In some embodiments, the at least one RNA aptamer reversal element binds to the at least one RNA aptamer with at least about 40% complementarity. In some embodiments, at least one RNA aptamer reversal element binds to the at least one RNA aptamer with at least about 45% complementarity. In some embodiments, the at least one RNA aptamer reversal element binds to the at least one RNA aptamer with at least about 50% complementarity. In some embodiments, the at least one RNA aptamer reversal element binds to the at least one RNA aptamer with at least about 55% complementarity. In some embodiments, at least one RNA aptamer reversal element binds to the at least one RNA aptamer with at least about 60% complementarity. In some embodiments, the at least one RNA aptamer reversal element binds to the at least one RNA aptamer with at least about 65% complementarity. In some embodiments, the at least one RNA aptamer reversal element binds to the at least one RNA aptamer with at least about 70% complementarity. In some embodiments, at least one RNA aptamer reversal element binds to the at least one RNA aptamer with at least about 75% complementarity. In some embodiments, the at least one RNA aptamer reversal element binds to the at least one RNA aptamer with at least about 80% complementarity. In some embodiments, the at least one RNA aptamer reversal element binds to the at least one RNA aptamer with at least about 85% complementarity. In some embodiments, at least one RNA aptamer reversal element binds to the at least one RNA aptamer with at least about 90% complementarity. In some embodiments, the at least one RNA aptamer reversal element binds to the at least one RNA aptamer with at least about 95% complementarity.

In some embodiments, the single-stranded nucleic acid molecule comprises at least two RNA aptamer reversal elements that are complementary to at least a portion of a single RNA aptamer having anti-coagulation activity. In some embodiments, the nucleic acid comprises at least two RNA aptamer reversal elements that are complementary to at least a portion of two different RNA aptamers having anti-coagulation activity.

In some embodiments, the at least two RNA aptamer reversal elements are separated by a linker region. In some embodiments, the linker region comprises from about 1 to about 50 nucleotides. In some embodiments, the linker region comprises from about 1 to about 40 nucleotides. In some embodiments, the linker region comprises from about 1 to about 30 nucleotides. In some embodiments, the linker region comprises from about 1 to about 20 nucleotides. In some embodiments, the linker region comprises from about 1 to about 10 nucleotides. In some embodiments, the linker region comprises from about 10 to about 50 nucleotides. In some embodiments, the linker region comprises from about 20 to about 50 nucleotides. In some embodiments, the linker region comprises from about 30 to about 50 nucleotides. In some embodiments, the linker region comprises from about 40 to about 50 nucleotides. In some embodiments, the at least two RNA aptamer reversal elements are continuous and not separated by a linker region.

In some embodiments, the linker region is 2 nucleotides in length, 3 nucleotides in length, 4 nucleotides in length, 5 nucleotides in length, 6 nucleotides in length, 7 nucleotides in length, 8 nucleotides in length, 9 nucleotides in length, 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, 31 nucleotides in length, 32 nucleotides in length, 33 nucleotides in length, 34 nucleotides in length, 35 nucleotides in length, 36 nucleotides in length, 37 nucleotides in length, 38 nucleotides in length, 39 nucleotides in length, 40 nucleotides in length, 41 nucleotides in length, 42 nucleotides in length, 43 nucleotides in length, 44 nucleotides in length, 45 nucleotides in length, 46 nucleotides in length, 47 nucleotides in length, 47 nucleotides in length, 49 nucleotides in length, or 50 nucleotides in length.

In some embodiments, the anti-coagulation activity of the RNA aptamer comprises inhibition of one or more of Factor XIIa, Factor XIIIa, Factor XIa, Factor IXa, Factor Xa, and/or von Willebrand factor. In some embodiments, the anti-coagulation activity of the RNA aptamer comprises thrombin inhibition. In some embodiments, the RNA aptamer is capable of binding exosite 1 of thrombin. In some embodiments, the RNA aptamer is capable of binding exosite 2 of thrombin. In some embodiments, the RNA aptamer is an anti-thrombin RNA_{R9D-14T} aptamer or a derivative thereof. In some embodiments, the RNA aptamer comprises an anti-thrombin Toggle-25t RNA aptamer or a derivative thereof.

In some embodiments, the at least one RNA aptamer reversal element comprises a sequence that is at least 80% identical to SEQ ID NOs: 1 or 2. In some embodiments, the at least one RNA aptamer reversal element comprises a sequence that is at least 85% identical to SEQ ID NOs: 1 or 2. In some embodiments, the at least one RNA aptamer reversal element comprises a sequence that is at least 90% identical to SEQ ID NOs: 1 or 2. In some embodiments, the at least one RNA aptamer reversal element comprises a sequence that is at least 91% identical to SEQ ID NOs: 1 or 2. In some embodiments, the at least one RNA aptamer reversal element comprises a sequence that is at least 92% identical to SEQ ID NOs: 1 or 2. In some embodiments, the at least one RNA aptamer reversal element comprises a sequence that is at least 93% identical to SEQ ID NOs: 1 or 2. In some embodiments, the at least one RNA aptamer reversal element comprises a sequence that is at least 94% identical to SEQ ID NOs: 1 or 2. In some embodiments, the at least one RNA aptamer reversal element comprises a sequence that is at least 95% identical to SEQ ID NOs: 1 or 2. In some embodiments, the at least one RNA aptamer reversal element comprises a sequence that is at least 96% identical to SEQ ID NOs: 1 or 2. In some embodiments, the at least one RNA aptamer reversal element comprises a sequence that is at least 97% identical to SEQ ID NOs: 1 or 2. In some embodiments, the at least one RNA aptamer reversal element comprises a sequence that is at least 98% identical to SEQ ID NOs: 1 or 2. In some embodiments, the at least one RNA aptamer reversal element comprises a sequence that is at least 99% identical to SEQ ID NOs: 1 or 2.

In some embodiments, the nucleic acid comprises a sequence that is at least 80% identical to any of SEQ ID NOs: 3-10. In some embodiments, the nucleic acid comprises a sequence that is at least 85% identical to any of SEQ ID NOs: 3-10. In some embodiments, the nucleic acid comprises a sequence that is at least 90% identical to any of SEQ ID NOs: 3-10. In some embodiments, the nucleic acid comprises a sequence that is at least 91% identical to any of SEQ ID NOs: 3-10. In some embodiments, the nucleic acid comprises a sequence that is at least 92% identical to any of SEQ ID NOs: 3-10. In some embodiments, the nucleic acid comprises a sequence that is at least 93% identical to any of SEQ ID NOs: 3-10. In some embodiments, the nucleic acid comprises a sequence that is at least 94% identical to any of SEQ ID NOs: 3-10. In some embodiments, the nucleic acid comprises a sequence that is at least 95% identical to any of SEQ ID NOs: 3-10. In some embodiments, the nucleic acid comprises a sequence that is at least 96% identical to any of SEQ ID NOs: 3-10. In some embodiments, the nucleic acid comprises a sequence that is at least 97% identical to any of SEQ ID NOs: 3-10. In some embodiments, the nucleic acid comprises a sequence that is at least 98% identical to any of SEQ ID NOs: 3-10. In some embodiments, the nucleic acid comprises a sequence that is at least 99% identical to any of SEQ ID NOs: 3-10.

In some embodiments, a nucleic acid molecule comprising an RNA aptamer reversal element(s) is a DNA molecule, a RNA molecule, an O-methyl RNA molecule, a fluoro-modified RNA molecule, a PNA molecule, an LNA molecule, or a combination or derivative thereof. In some embodiments, the at least one nucleic acid antidote binds to at least a portion of any of the single-stranded nucleic acid molecules described herein in a reverse complementary manner. In some embodiments, the at least one nucleic acid antidote binds to the least one nucleic acid aptamer of any of the single-stranded nucleic acid molecules described herein to counteract the anti-coagulant activity.

Embodiments of the present disclosure also include a system or kit for modulating coagulation. In accordance with these embodiments, the kit or system includes any of the single-stranded nucleic acid molecules described herein comprising at least one RNA aptamer reversal element that is complementary to at least a portion of an RNA aptamer having anti-coagulation activity, and the RNA aptamer having anticoagulation activity. As described further herein, the single-stranded nucleic acid molecules comprising an RNA aptamer reversal element(s) counteract the anticoagulant activity of the RNA aptamers. As would be recognized by one of ordinary skill in the art based on the present disclosure, the system or kit can be used for treating a subject or patient in accordance with a treatment regimen to modulate blood coagulation in the context of a surgical procedure and/or to treat a disease condition.

Embodiments of the present disclosure also include a vector comprising any of the nucleic acid molecules comprising an RNA aptamer reversal element described herein. In some embodiments, the vector is an expression vector. Examples of an expression vector include, but are not limited to, a plasmid, a cosmid, a viral vector, an RNA vector, or a linear or circular DNA or RNA molecule. As would be recognized by one of ordinary skill in the art based on the present disclosure, the term “construct” refers to any polynucleotide that contains a recombinant nucleic acid. A construct may be present in a vector (e.g., a bacterial vector, a viral vector) or may be integrated into a genome. A “vector” is a nucleic acid molecule that is capable of transporting another nucleic acid. Vectors may be, for example, plasmids, cosmids, viruses, a RNA vector, or a linear or circular DNA or RNA molecule that may include chromosomal, non-chromosomal, semi-synthetic or synthetic nucleic acids. Exemplary vectors are those capable of autonomous replication (episomal vector) and/or expression of nucleic acids to which they are linked (expression vectors). Additionally, as used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. A viral vector may be DNA (e.g., an Adenovirus or Vaccinia virus) or RNA-based including an oncolytic virus vector (e.g., VSV), replication competent or incompetent. Once transformed into a suitable host, the vector may replicate and function independently of the host genome, or may, in some instances, integrate into the genome itself. In the present specification, “plasmid,” “expression plasmid,” “virus” and “vector” are often used interchangeably.

Therapeutic methods and compositions. Embodiments of the present disclosure also include a therapeutic composition comprising any of the nucleic acid molecules described herein and a pharmaceutically acceptable excipient, solvent, carrier, or diluent. In some embodiments, the RNA aptamers and/or RNA aptamer antidotes/reversal agents are administered to a subject as a therapeutic composition to treat a condition or disease (e.g., a disease or condition involving blood coagulation). In some embodiments, a “therapeutically effective amount (or dose)” or “effective amount (or dose)” of a therapeutic composition refers to that amount of the composition (or one or more active agents in the composition) sufficient to result in amelioration or modulation of one or more symptoms of the disease being treated. The precise amount will depend upon numerous factors, including but not limited to, the activity of the composition, the method of delivery employed, the immune stimulating ability of the composition, the intended patient and patient considerations, or the like, and can readily be determined by one of ordinary skill in the art. A therapeutic effect may include, directly or indirectly, the reduction of one or more symptoms of a disease (e.g., modulation of blood coagulation).

Embodiments of the present disclosure also include a method of modulating coagulation in a subject in need thereof. In accordance with these embodiments, the method includes administering any of the therapeutic compositions described herein to a subject. In some embodiments, the administration of a therapeutic compositions described herein to a subject is performed after the subject has been administered a composition comprising an RNA aptamer having anti-coagulation activity. As would be recognized by one of ordinary skill in the art based on the present disclosure, the term “treatment,” “treating” or “ameliorating” refers to medical management of a disease, disorder, or condition of a subject (e.g., patient), which may be therapeutic, prophylactic/preventative, or a combination treatment thereof. A treatment may improve or decrease the severity at least one symptom of a disease, delay worsening or progression of a disease, or delay or prevent onset of additional associated diseases. A treatment can also be modulatory, such as modulating anticoagulation activity in a subject.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 3. MATERIALS AND METHODS

Amplification of G-Block sequence or plasmid DNA were performed. Reaction buffer, forward and reverse primers, dNTP, DNA polymerase and nuclease-free water was added to a PCR tube in the concentrations shown in Table 1. DNA Polymerase was added last and the sample was pipetted to mix.

TABLE 1
PCR concentrations and volumes.

| | Final Concentration |
|---|---|
| Reaction Buffer (x) | 1 |
| Forward Primer (μM) | 0.5 |
| Reverse Primer (μM) | 0.5 |
| dNTP (mM) | 0.2 |
| DNA Template (μl/50 μl) | 2 |
| DNA Polymerase (unit/μl) | 0.02 |
| Nuclease-free Water | To desired volume |

Following PCR, a small volume of the amplified DNA sample (˜2-5 μl) was used for testing in an 1% agarose gel. The DNA was run beside a 1 kb ladder (Promega) at 150V for 30 minutes. The gel was then viewed under UV light (ProteinSimple instrument). If the proper band size was observed, the remaining DNA sample was then used for in-solution purification using the GFX DNA Purification Kit (GE Healthcare). About 1 μl of the purified solution was used for analysis using the Nanodrop 3000c spectrophotometer (ThermoFisher Scientific). The concentration was recorded for further use and the DNA was kept and labeled (2H-XXXX) for later transcription.

Non-modified transcription was then performed. All contents found in Table 2 below, excluding the RNA T7 polymerase, were mixed in a PCR tube. It should be noted that the DTT used was mixed in-lab to ensure freshness. Lastly, the RNA T7 polymerase was added and the sample was pipetted to mix. The sample was synthesized on ice to try and slow down enzymes, such as RNase, which have a negative impact on the production of RNA. Once all components were added and mixed, the sample was placed in the thermocycler and incubated at 37° C. for 4-16 hours and then held at 4° C.

TABLE 2
Non-Modified Transcription contents and concentrations.

| | Final concentration |
|---|---|
| Transcription NEB buffer (x) | 1 |
| Fresh DTT (mM) | 5 |
| NTPs (mM each) | 2.5 |
| DNA Template (ng/uL) | 10 |
| RNA T7 Polymerase (μl/50 μl) | 2.5 |
| Nuclease-free water | To desired volume |

For fluoro-modified transcription, the same protocol as above was followed, but using the contents found in Table 3 below:

TABLE 3
Fluoro-Modified Transcription contents and concentrations.

| | Final concentration |
|---|---|
| Transcription NEB buffer (x) | 1 |
| Fresh DTT (mM) | 10 |
| Modified NTPs (mM each) | 2.5 |
| DNA Template (ng/uL) | 10 |
| Mutant RNA T7 Polymerase (μl/50 μl) | 2.5 |
| Nuclease-free water | To desired volume |

For fluoro-modified transcription with native RNA T7 polymerase, the same protocol as above was followed, but using the contents found in Table 4 below:

TABLE 4
Fluoro-Modified Transcription contents and concentrations.

| | Final concentration |
|---|---|
| Transcription NEB buffer (x) | 1 |
| Fresh DTT (mM) | 10 |
| Modified NTPs (mM each) | 2.5 |
| DNA Template (ng/uL) | 10 |
| MnCl₂ (mM) | 0.5 |
| RNA T7 Polymerase (μl/50 μl) | 2.5 |
| Nuclease-free water | To desired volume |

Following transcription, a small volume of the sample (˜5 μl) was used for viewing in a 6% acrylamide denaturing gel. The sample was run beside a nucleic acid marker at 20 W for 1 hour. The gel was then viewed under UV light to check for correct length. If the sequence length was correct and a nice band was present, the sample was then purified using the Monarch RNA Clean-Up kit. About 31 μl of elution buffer was used for purification and 1 μl of sample was used for analysis with the Nanodrop 3000c. The optical density (A260) was recorded and Beer's Law was used to calculate the molar concentration of the sample.

The RNA sample diluted in 1× folding buffer was then heat annealed by heating at 95° C. for 5 minutes followed by cooling at room temperature for 30 minutes. A 1× folding buffer was used to dilute the sample to the desired volume for further use.

For specificity tests, heat-annealed RNA origami or DNA weave tile (5 pmol) was dissolved in 1× annealing buffer and incubated with protein (25 pmol) at 37° C. for 1 hr. The samples were tested by 6% native acrylamide gel electrophoresis in 1×TBE as running buffer at 150 V for 3-6 hr. The gels were stained with Ethidium bromide for nucleic acid staining and visualized under a UV lamp. Then, the gels were further stained with Coomassie blue for protein staining and imaged with the ProteinSimple instrument.

For folding RNA origami, the non-modified and modified RNA origami were dissolved 1× folding buffer was heated at 95° C. for 5 min and let cool down at room temperature for 30 min. Finally, 1× buffer was added into the folded RNA origami to get the desired concentration. The folded RNA origami (1 μl, 5 μM) was mixed with RNase A (1 μl of 10 and 500 μg/ml) or human plasma (3 μl) and incubated at 37° C. for various time course from 10 min to 24 hr. The integrity of RNA origami was characterized by denaturing gel electrophoresis. For the denaturing gel electrophoresis, the gels were pre-run at 20 W for 30 min, and the samples were run at 20 W for 1 hr. Finally, the gels were stained with Ethidium bromide for nucleic acid staining. The nucleic acid bands were visualized under UV lamp of ProteinSimple instrument.

Anticoagulation activity of RNA origami anticoagulants using Activated Partial Thromboplastin Time (APTT) assay. To test the anticoagulation activity of the RNA origami, an ST4 Coagulometer (Diagnostica Stago) was used to perform aPTT coagulation assays. 50 μL of pooled human blood plasma (George King Bio-Medical, Inc.) and 50 μL of aPTT reagent were incubated for 300 seconds. RNA origami (16.67 μL) was then added and allowed to further incubate for 300 seconds. Fifty microliters of CaCl2 was added to activate the clot formation. The clotting times were recorded.

Anticoagulation activity of RNA origami anticoagulants using Prothrombin time (PT) assay. To test the extrinsic pathway-based anticoagulation activity of the RNA origami, an ST4 Coagulometer (Diagnostica Stago) was used to perform PT coagulation assays. PT assay is a test of extrinsic coagulation pathway and uses to evaluate the coagulation times of pooled human blood plasma with RNA origami anticoagulants. About 16.67 μL of 1-4 μM RNA origami samples were tested in 50 μL of pooled human blood plasma and 100 μL PT reagent. The clotting times were recorded.

Reversal activity of DNA antidote using APTT assay. Anticoagulant activity of 2HF-2211 was reversed using single-molecule DNA antidote strands. 50 μL of pooled human blood plasma (George King Bio-Medical, Inc.) and 50 μL of aPTT reagent were incubated for 300 seconds. A 13.67 μL of 2HF-2211 was added and incubated for 300 seconds. Then, 3 μL of single-molecule DNA antidote was added and incubated for an additional 300 seconds. For the control sample, 3 μL of annealing buffer was added in place of the antidote solution. For rapid action, incubation times of DNA antidotes were varied from 30-600 seconds. After that, 50 μL of CaCl2 were added to activate the clot formation. The clotting times were recorded.

Efficacy of RNA origami anticoagulant in murine models. Male C57BL/6J mice (8-10-week-old) were anesthetized using up to 5% isoflurane supplied in medical grade oxygen administered through inhalation in a chamber. They will then be moved to a scavenging breathing circuit with nose cone and maintained at 1-3% isoflurane. The animals' body temperature will be maintained with a heating pad until recovery from anesthesia. RNA anticoagulant (0.5-3 mg/kg) was injected intravenously and allowed to circulate for 5 minutes. Liver laceration injury was performed, and blood loss was collected.

Reversal activity of single-molecule DNA antidote in murine models. Male C57BL/6J mice (8-10-week-old) were anesthetized. 1 mg/kg RNA anticoagulant was injected intravenously and allowed to circulate for 5 minutes. Followed by various concentration of antidote (5 or 10 equivalent) or saline (vesicle) was administered by intravenous injection and allowed to circulate for 5 minutes prior to injury. Liver laceration injury was performed.

### 4. SEQUENCES

The following nucleic acid sequences are provided by the present disclosure and referenced herein.

RNA aptamer reversal element: GTCTGCCTCGTCATTGGCT (SEQ ID NO: 1)

RNA aptamer reversal element: GGGTAAGTACTTCAGCTTTGTTCCC (SEQ ID NO: 2)

Single-stranded nucleic acid molecule comprising an RNA aptamer reversal element: GGGTAAGTACTTCAGCTTTGTTCCCGTCTGCCTCGTACATTGGCT (SEQ ID NO: 3)

Single-stranded nucleic acid molecule comprising an RNA aptamer reversal element:

(SEQ ID NO: 4)
GGGTAAGTACTTCAGCTTTGTTCCCTCACCTATTACGTCTGCCTCGTAC
ATTGGCT

Single-stranded nucleic acid molecule comprising an RNA aptamer reversal element:

(SEQ ID NO: 5)
GGGTAAGTACTTCAGCTTTGTTCCCTCCACCTCACTCACCTATTACGTC
TGCCTCGTACATTGGCT

Single-stranded nucleic acid molecule comprising an RNA aptamer reversal element:

(SEQ ID NO: 6)
GGGTAAGTACTTCAGCTTTGTTCCCACATCACCACTCCACCTCACTCAC
CTATTACGTCTGCCTCGTACATTGGCT

Single-stranded nucleic acid molecule comprising an RNA aptamer reversal element: GTCTGCCTCGTACATTGGCTGGGTAAGTACTTCAGCTTTGTTCCC (SEQ ID NO: 7)

Single-stranded nucleic acid molecule comprising an RNA aptamer reversal element:

(SEQ ID NO: 8)
GTCTGCCTCGTACATTGGCTTCCACTTCACGGGTAAGTACTTCAGCTTT
GTTCCC

Single-stranded nucleic acid molecule comprising an RNA aptamer reversal element:

(SEQ ID NO: 9)
GTCTGCCTCGTACATTGGCTTCCACTTCACTCATCTATTACGGGTAAGT
ACTTCAGCTTTGTTCCC

Single-stranded nucleic acid molecule comprising an RNA aptamer reversal element:

(SEQ ID NO: 10)
GTCTGCCTCGTACATTGGCTACATCACCACTCCACTTCACTCATCTATT
ACGGGTAAGTACTTCAGCTTTGTTCCC
RNA-NNNN
(SEQ ID NO: 11)
GAGAUCGAGCGACUUCCGACUUCGGUCGGGAGUCGGGCUAGUCAU
CUUCGGAUGAUUAGCCGCUGGUGAAGCCUCCACGCCAGCCUCGGUCUCC
CGCAGUAGGAUCGGACUGAAGGAGGCACGGUCCCAGCCGAAGUGUCUUG
CUUCGGCAAGGCACUUUGGCUGCUAGACUGGCUGGCUUCGGCCAGCUAG
UUUAGGAUUCUAUUGC
RNA-12NN
(SEQ ID NO: 12)
GGGAGAUCGAGCGACUUCCGACUUCGGUCGGGAGUCGGGCUAGUCAU
CGGGAACAAAGCUGAAGUACUUACCCGAUGAUUAGCCGCUGGUGAAGCC
UCCACGCCAGCCUCGGUCUCCCGCAGUAGGAUCGGACUGAAGGAGGCAC
GGUCCCAGCCGAAGUGUCUGGCGGUCGAUCACACAGUUCAAACGUAAUA
AGCCAAUGUACGAGGCAGACGACUCGCCAGGCACUUUGGCUGCUAGACU
GGCUGGCUUCGGCCAGCUAGUUUAGGAUUCUAUUGC
RNA-1N2N
(SEQ ID NO: 13)
GGGAGAUCGAGCGACUUCCGACGGGAACAAAGCUGAAGUACUUACCC
GUCGGGAGUCGGGCUAGUCAUCUUCGGAUGAUUAGCCGCUGGUGAAGCC
UCCACGCCAGCCUCGGUCUCCCGCAGUAGGAUCGGACUGAAGGAGGCAC
GGUCCCAGCCGAAGUGUCUGGCGGUCGAUCACACAGUUCAAACGUAAUA
AGCCAAUGUACGAGGCAGACGACUCGCCAGGCACUUUGGCUGCUAGACU
GGCUGGCUUCGGCCAGCUAGUUUAGGAUUCUAUUGC
RNA-2NN1
(SEQ ID NO: 14)
GGGAGAUCGAGCGACUUCCGACUUCGGUCGGGAGUCGGGCUAGUCAU
CUUCGGAUGAUUAGCCGCUGGUGAAGCCUCCACGCCAGCCUCGGUCUCC
CGCAGUAGGAUCGGACUGAAGGAGGCACGGUCCCAGCCGAAGUGUCUUG
CGGGAACAAAGCUGAAGUACUUACCCGCAAGGCACUUUGGCUGCUAGAC
UGGCUGGCGGCGGUCGAUCACACAGUUCAAACGUAAUAAGCCAAUGUAC
GAGGCAGACGACUCGCCGCCAGCUAGUUUAGGAUUCUAUUGC
Fss12
(SEQ ID NO: 15)
GGGAACAAAGCUGAAGUACUUACCCACCUUACCACUCCACCUCACUC
ACCUAUUACGGCGGUCGAUCACACAGUUCAAACGUAAUAAGCCAAUGUA
CGAGGCAGACGACUCGCC
2H-2211a
(SEQ ID NO: 16)
GGGAGAUCGAGCGACUUCCGACUCUGGCGGUCGAUCACACAGUUCAA
ACGUAAUAAGCCAAUGUACGAGGCAGACGACUCGCCAGAGUCGGGAGUC
GGGCUAGUCAUCAGGCACGGGAACAAAGCUGAAGUACUUACCCGUGCCU
GAUGAUUAGCCGCUGGUGAAGCCUCCACGCCAGCCUCGGUCUCCCGCAG
UAGGAUCGGACUGAAGGAGGCACGGUCCCAGCCGAAGUGUCUUGCGGGA
ACAAAGCUGAAGUACUUACCCGCAAGGCACUUUGGCUGCUAGACUGGCU
GGCGGCGGUCGAUCACACAGUUCAAACGUAAUAAGCCAAUGUACGAGGC
AGACGACUCGCCGCCAGCUAGUUUAGGAUUCUAUUGC
2H-2211b
(SEQ ID NO: 17)
GGGAGAUCGAGCGACUUCCGACUCUGGCGGUCGAUCACACAGUUCAA
ACGUAAUAAGCCAAUGUACGAGGCAGACGACUCGCCAGAGUCGGGAGUC
GGGCUAGUCAUCAGGCACGGGAACAAAGCUGAAGUACUUACCCGUGCCU
GAUGAUUAGCCGCUGGUGAAGCCUCCACGCCAGCCUCGGUCUCCCGAUC
UAGGAUCGGACUGAAGGAGGCACGGUCCCAGCCGAAGUGUCUUGCGGGA
ACAAAGCUGAAGUACUUACCCGCAAGGCACUUUGGCUGCUAGACUGGCU
GGCGGCGGUCGAUCACACAGUUCAAACGUAAUAAGCCAAUGUACGAGGC
AGACGACUCGCCGCCAGCUAGUUUAGGAUUCUAGAUC
3H-2NN1
(SEQ ID NO: 18)
GGAAAUGAUGCCGAGUUGACGCUUCGGCGUCAGCUCGCCCUGUGGCC
UAGUUCGCUAGGUCACAGACAUCUUGGCGUUCGCGCCAGGAUGUCUCGC
CCAAUUCCGUAGGGCGAGGGUAGCCAAAUCCAGAGGCUAGCAUUAUUUC
CGAUCUAGGAUCGCGUUGAGAACUGGAUACUCAACAGCGGUAAACGGAA
AACCGCUCAGCCGAAGUGUCUUGCGGGAACAAAGCUGAAGUACUUACCC
GCAAGGCACUUUGGCUGGCCACGCGUCGUAUUCGUACGGCGCGUGCUAG
ACUGGCUGGCGGCGGUCGAUCACACAGUUCAAACGUAAUAAGCCAAUGU
ACGAGGCAGACGACUCGCCGCCAGCUAGUUUAGGAUUCUAGAUC
4H-2NN1
(SEQ ID NO: 19)
GGAAAUGAUGCCGAGUUGACGCUUCGGCGUCAGCUCGCCCUGUGGCC
UAGUUCGCUAGGUCACAGCCGACCAUUGCGUUUCGACGCAGUGGUCACA
UCUUGGCGUUCGCGCCAGGAUGUCUCGCCCAAUUCCGUAGGGCGAGGGG
ACCCAAAUCCCUAGGGUCGGUAGCCAAAUCCAGAGGCUAGCAUUAUUUC
CGAUCUAGGAUCGCGUUGAGAACUGGAUACUCAACCGUGGCAUAAAGGG
AUAAUGCCAAGCGGUAAACGGAAAACCGCUCAGCCGAAGUGUCUUGCGG
GAACAAAGCUGAAGUACUUACCCGCAAGGCACUUUGGCUGCGUGGCGUU
ACAGUUCGCUGUGACGCCAGCCACGCGUCGUAUUCGUACGGCGCGUGCU
AGACUGGCUGGCGGCGGUCGAUCACACAGUUCAAACGUAAUAAGCCAAU
GUACGAGGCAGACGACUCGCCGCCAGCUAGUUUAGGAUUCUAGAUC

Gblock sequences are provided below (T7 promoters are bolded).

2H-DNAGblock-NNNN
(SEQ ID NO: 20)
CACTTTCAGCCCTCTTATCCTCGGCGGATCCTTCTAATACGACTCACTA
TAGGGAGATCGAGCGACTTCCGACTTCGGTCGGGAGTCGGGCTAGTCAT
CTTCGGATGATTAGCCGCTGGTGAAGCCTCCACGCCAGCCTCGGTCTCC
CGCAGTAGGATCGGACTGAAGGAGGCACGGTCCCAGCCGAAGTGTCTTG
CTTCGGCAAGGCACTTTGGCTGCTAGACTGGCTGGCTTCGGCCAGCTAG
TTTAGGATTCTATTGC
2H-DNAGblock-12NN
(SEQ ID NO: 21)
CACTTTCAGCCCTCTTATCCTCGGCGGATCCTTCTAATACGACTCACTA
TAGGGAGATCGAGCGACTTCCGACTTCGGTCGGGAGTCGGGCTAGTCAT
CGGGAACAAAGCTGAAGTACTTACCCGATGATTAGCCGCTGGTGAAGCC
TCCACGCCAGCCTCGGTCTCCCGCAGTAGGATCGGACTGAAGGAGGCAC
GGTCCCAGCCGAAGTGTCTGGCGGTCGATCACACAGTTCAAACGTAATA
AGCCAATGTACGAGGCAGACGACTCGCCAGGCACTTTGGCTGCTAGACT
GGCTGGCTTCGGCCAGCTAGTTTAGGATTCTATTGC
2H-DNAGblock-1N2N
(SEQ ID NO: 22)
CACTTTCAGCCCTCTTATCCTCGGCGGATCCTTCTAATACGACTCACTA
TAGGGAGATCGAGCGACTTCCGACGGGAACAAAGCTGAAGTACTTACCC
GTCGGGAGTCGGGCTAGTCATCTTCGGATGATTAGCCGCTGGTGAAGCC
TCCACGCCAGCCTCGGTCTCCCGCAGTAGGATCGGACTGAAGGAGGCAC
GGTCCCAGCCGAAGTGTCTGGCGGTCGATCACACAGTTCAAACGTAATA
AGCCAATGTACGAGGCAGACGACTCGCCAGGCACTTTGGCTGCTAGACT
GGCTGGCTTCGGCCAGCTAGTTTAGGATTCTATTGC
2H-DNAGblock-2NN1
(SEQ ID NO: 23)
CACTTTCAGCCCTCTTATCCTCGGCGGATCCTTCTAATACGACTCACTA
TAGGGAGATCGAGCGACTTCCGACTTCGGTCGGGAGTCGGGCTAGTCAT
CTTCGGATGATTAGCCGCTGGTGAAGCCTCCACGCCAGCCTCGGTCTCC
CGCAGTAGGATCGGACTGAAGGAGGCACGGTCCCAGCCGAAGTGTCTTG
CGGGAACAAAGCTGAAGTACTTACCCGCAAGGCACTTTGGCTGCTAGAC
TGGCTGGCGGCGGTCGATCACACAGTTCAAACGTAATAAGCCAATGTAC
GAGGCAGACGACTCGCCGCCAGCTAGTTTAGGATTCTATTGC
DNAGblock-Fss12
(SEQ ID NO: 24)
CACTTTCAGCCCTCTTATCCTCGGCGGATCCTTCTAATACGACTCACTA
TAGGGAACAAAGCTGAAGTACTTACCCACCTTACCACTCCACCTCACTC
ACCTATTACGGCGGTCGATCACACAGTTCAAACGTAATAAGCCAATGTA
CGAGGCAGACGACTCGCC
2H-DNAGblock-2211a
(SEQ ID NO: 25)
CACTTTCAGCCCTCTTATCCTCGGCGGATCCTTCTAATACGACTCACTA
TAGGGAGATCGAGCGACTTCCGACTCTGGCGGTCGATCACACAGTTCAA
ACGTAATAAGCCAATGTACGAGGCAGACGACTCGCCAGAGTCGGGAGTC
GGGCTAGTCATCAGGCACGGGAACAAAGCTGAAGTACTTACCCGTGCCT
GATGATTAGCCGCTGGTGAAGCCTCCACGCCAGCCTCGGTCTCCCGCAG
TAGGATCGGACTGAAGGAGGCACGGTCCCAGCCGAAGTGTCTTGCGGGA
ACAAAGCTGAAGTACTTACCCGCAAGGCACTTTGGCTGCTAGACTGGCT
GGCGGCGGTCGATCACACAGTTCAAACGTAATAAGCCAATGTACGAGGC
AGACGACTCGCCGCCAGCTAGTTTAGGATTCTATTGC
2H-DNAGblock-2211b
(SEQ ID NO: 26)
CACTTTCAGCCCTCTTATCCTCGGCGGATCCTTCTAATACGACTCACTA
TAGGGAGATCGAGCGACTTCCGACTCTGGCGGTCGATCACACAGTTCAA
ACGTAATAAGCCAATGTACGAGGCAGACGACTCGCCAGAGTCGGGAGTC
GGGCTAGTCATCAGGCACGGGAACAAAGCTGAAGTACTTACCCGTGCCT
GATGATTAGCCGCTGGTGAAGCCTCCACGCCAGCCTCGGTCTCCCGATC
TAGGATCGGACTGAAGGAGGCACGGTCCCAGCCGAAGTGTCTTGCGGGA
ACAAAGCTGAAGTACTTACCCGCAAGGCACTTTGGCTGCTAGACTGGCT
GGCGGCGGTCGATCACACAGTTCAAACGTAATAAGCCAATGTACGAGGC
AGACGACTCGCCGCCAGCTAGTTTAGGATTCTAGATC
3H-DNAGblock-2NN1
(SEQ ID NO: 27)
CGGCCAGTGAATTCGAGCTCGGTACCCGGGAGATCTCACTTTCAGCCCT
CTTATCCTCGGCCGATCCTTCTAATACGACTCACTATAGGAAATGATGC
CGAGTTGACGCTTCGGCGTCAGCTCGCCCTGTGGCCTAGTTCGCTAGGT
CACAGACATCTTGGCGTTCGCGCCAGGATGTCTCGCCCAATTCCGTAGG
GCGAGGGTAGCCAAATCCAGAGGCTAGCATTATTTCCGATCTAGGATCG
CGTTGAGAACTGGATACTCAACAGCGGTAAACGGAAAACCGCTCAGCCG
AAGTGTCTTGCGGGAACAAAGCTGAAGTACTTACCCGCAAGGCACTTTG
GCTGGCCACGCGTCGTATTCGTACGGCGCGTGCTAGACTGGCTGGCGGC
GGTCGATCACACAGTTCAAACGTAATAAGCCAATGTACGAGGCAGACGA
CTCGCCGCCAGCTAGTTTAGGATTCTAGATC
4H-DNAGblock-2NN1
(SEQ ID NO: 28)
CGGCCAGTGAATTCGAGCTCGGTACCCGGGAGATCTCACTTTCAGCCCT
CTTATCCTCGGCCGATCCTTCTAATACGACTCACTATAGGAAATGATGC
CGAGTTGACGCTTCGGCGTCAGCTCGCCCTGTGGCCTAGTTCGCTAGGT
CACAGCCGACCATTGCGTTTCGACGCAGTGGTCACATCTTGGCGTTCGC
GCCAGGATGTCTCGCCCAATTCCGTAGGGCGAGGGGACCCAAATCCCTA
GGGTCGGTAGCCAAATCCAGAGGCTAGCATTATTTCCGATCTAGGATCG
CGTTGAGAACTGGATACTCAACCGTGGCATAAAGGGATAATGCCAAGCG
GTAAACGGAAAACCGCTCAGCCGAAGTGTCTTGCGGGAACAAAGCTGAA
GTACTTACCCGCAAGGCACTTTGGCTGCGTGGCGTTACAGTTCGCTGTG
ACGCCAGCCACGCGTCGTATTCGTACGGCGCGTGCTAGACTGGCTGGCG
GCGGTCGATCACACAGTTCAAACGTAATAAGCCAATGTACGAGGCAGACG
ACTCGCCGCCAGCTAGTTTAGGATTCTAGATC

Plasmid sequence for 2H-2211b template is provided below (T7 promoters are bolded and DNA template region is italic)

pUC19_AMP⁺_2H-2211b
(SEQ ID NO: 29)
CATCAGGCGCCATTCGCCATTCAGGCTGCGCAACTGTTGGGAAGGGCG
ATCGGTGCGGGCCTCTTCGCTATTACGCCAGCTGGCGAAAGGGGGATGTGCTGCA
AGGCGATTAAGTTGGGTAACGCCAGGGTTTTCCCAGTCACGACGTTGTAAAACG
ACGGCCAGTGAATTCGAGCTCGGTACCCGGGAGATCTCACTTTCAGCCCTCTTAT
CCTCGGCCGATCCTTCTAATACGACTCACTATAGGGAGATCGAGCGACTTCCGAC
TCTGGCGGTCGATCACACAGTTCAAACGTAATAAGCCAATGTACGAGGCAGACGACTC
GCCAGAGTCGGGAGTCGGGCTAGTCATCAGGCACGGGAACAAAGCTGAAGTACTTAC
CCGTGCCTGATGATTAGCCGCTGGTGAAGCCTCCACGCCAGCCTCGGTCTCCCGATC
TAGGATCGGACTGAAGGAGGCACGGTCCCAGCCGAAGTGTCTTGCGGGAACAAAGCT
GAAGTACTTACCCGCAAGGCACTTTGGCTGCTAGACTGGCTGGCGGCGGTCGATCAC
ACAGTTCAAACGTAATAAGCCAATGTACGAGGCAGACGACTCGCCGCCAGCTAGTTTA
GGATTCTAGATCTCTCTAGAGTCGACCTGCAGGCATGCAAGCTTGGCGTAATCATG
GTCATAGCTGTTTCCTGTGTGAAATTGTTATCCGCTCACAATTCCACACAACATAC
GAGCCGGAAGCATAAAGTGTAAAGCCTGGGGTGCCTAATGAGTGAGCTAACTCA
CATTAATTGCGTTGCGCTCACTGCCCGCTTTCCAGTCGGGAAACCTGTCGTGCCA
GCTGCATTAATGAATCGGCCAACGCGCGGGGAGAGGCGGTTTGCGTATTGGGCG
CTCTTCCGCTTCCTCGCTCACTGACTCGCTGCGCTCGGTCGTTCGGCTGCGGCGAG
CGGTATCAGCTCACTCAAAGGCGGTAATACGGTTATCCACAGAATCAGGGGATA
ACGCAGGAAAGAACATGTGAGCAAAAGGCCAGCAAAAGGCCAGGAACCGTAAA
AAGGCCGCGTTGCTGGCGTTTTTCCATAGGCTCCGCCCCCCTGACGAGCATCACA
AAAATCGACGCTCAAGTCAGAGGTGGCGAAACCCGACAGGACTATAAAGATACC
AGGCGTTTCCCCCTGGAAGCTCCCTCGTGCGCTCTCCTGTTCCGACCCTGCCGCTT
ACCGGATACCTGTCCGCCTTTCTCCCTTCGGGAAGCGTGGCGCTTTCTCATAGCTC
ACGCTGTAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATCGTCTTG
AGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGC
CTAACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGGA
TCTCAAGAAGATCCTTTGATCTTTTCTACGGGGTCTGACGCTCAGTGGAACGAAA
ACTCACGTTAAGGGATTTTGGTCATGAGATTATCAAAAAGGATCTTCACCTAGAT
CCTTTTAAATTAAAAATGAAGTTTTAAATCAATCTAAAGTATATATGAGTAAACT
TGGTCTGACAGTTACCAATGCTTAATCAGTGAGGCACCTATCTCAGCGATCTGTC
TATTTCGTTCATCCATAGTTGCCTGACTCCCCGTCGTGTAGATAACTACGATACGG
GAGGGCTTACCATCTGGCCCCAGTGCTGCAATGATACCGCGAGACCCACGCTCA
CCGGCTCCAGATTTATCAGCAATAAACCAGCCAGCCGGAAGGGCCGAGCGCAGA
AGTGGTCCTGCAACTTTATCCGCCTCCATCCAGTCTATTAATTGTTGCCGGGAAG
CTAGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTTCCC
AACGATCAAGGCGAGTTACATGATCCCCCATGTTGTGCAAAAAAGCGGTTAGCT
CCTTCGGTCCTCCGATCGTTGTCAGAAGTAAGTTGGCCGCAGTGTTATCACTCAT
GGTTATGGCAGCACTGCATAATTCTCTTACTGTCATGCCATCCGTAAGATGCTTTT
CTGTGACTGGTGAGTACTCAACCAAGTCATTCTGAGAATAGTGTATGCGGCGACC
GAGTTGCTCTTGCCCGGCGTCAATACGGGATAATACCGCGCCACATAGCAGAACT
TTAAAAGTGCTCATCATTGGAAAACGTTCTTCGGGGCGAAAACTCTCAAGGATCT
TACCGCTGTTGAGATCCAGTTCGATGTAACCCACTCGTGCACCCAACTGATCTTC
AGCATCTTTTACTTTCACCAGCGTTTCTGGGTGAGCAAAAACAGGAAGGCAAAAT
GCCGCAAAAAAGGGAATAAGGGCGACACGGAAATGTTGAATACTCATACTCTTC
CTTTTTCAATATTATTGAAGCATTTATCAGGGTTATTGTCTCATGAGCGGATACAT
ATTTGAATGTATTTAGAAAAATAAACAAATAGGGGTTCCGCGCACATTTCCCCGA
AAAGTGCCACCTGACGTCTAAGAAACCATTATTATCATGACATTAACCTATAAAA
ATAGGCGTATCACGAGGCCCTTTCGTCTCGCGCGTTTCGGTGATGACGGTGAAAA
CCTCTGACACATGCAGCTCCCGGAGACGGTCACAGCTTGTCTGTAAGCGGATGCC
GGGAGCAGACAAGCCCGTCAGGGCGCGTCAGCGGGTGTTGGCGGGTGTCGGGGC
TGGCTTAACTATGCGGCATCAGAGCAGATTGTACTGAGAGTGCACCATATGCGGT
GTGAAATACCGCACAGATGCGTAAGGAGAAAATACCG

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

### 5. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

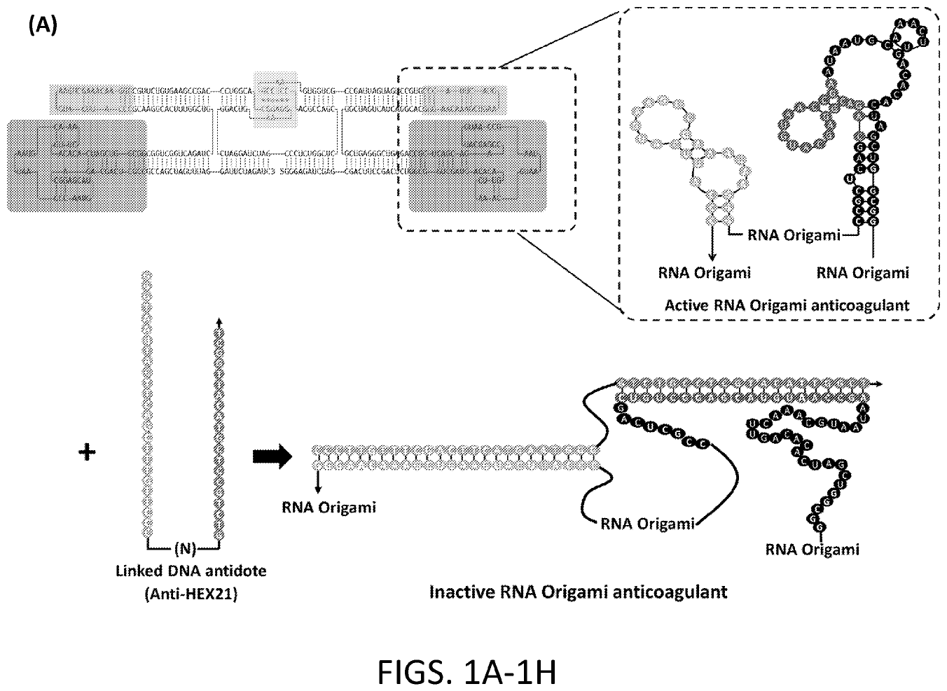

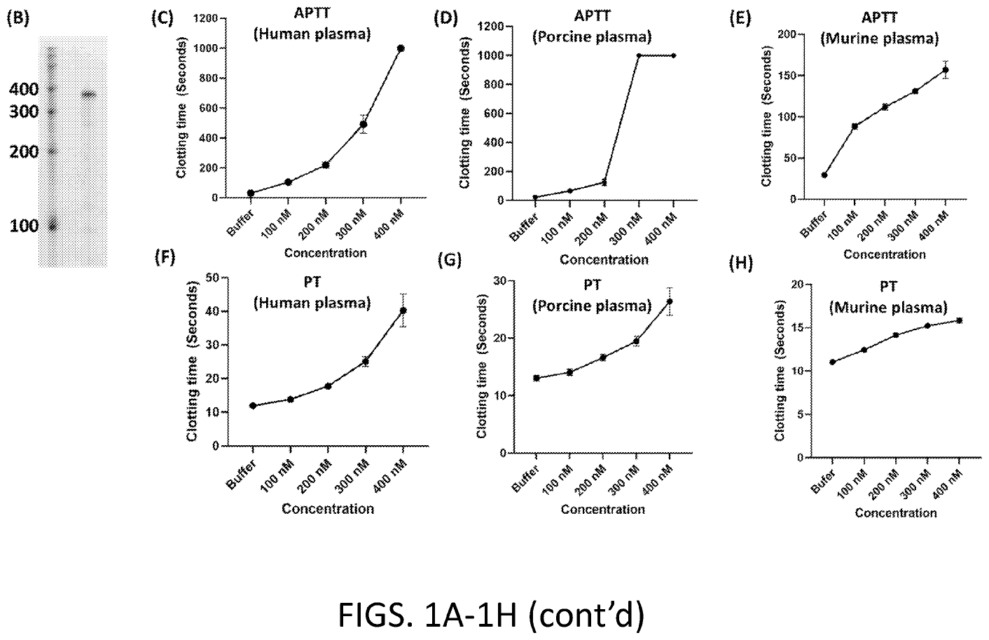

Anticoagulation activity of RNA origami. Single-stranded, self-folding RNA origami anticoagulant and single-molecule DNA antidote were produced, as shown in FIG. 1A. The RNA origami anticoagulant contains two copies of exosite-1 and exosite-2 binding RNA aptamers. Single-molecule DNA antidotes have sequences complimentary to RNA aptamers that served as reversal agent (antidote) for RNA origami anticoagulant. Eight single-molecule DNA antidotes were designed by varying length of linkers and rearranging position of antidote sequences. The RNA origami anticoagulant is 378 nucleotides produced by in vitro transcription. The size of RNA origami was characterized using denaturing polyacrylamide gel electrophoresis (FIG. 1B). To test anticoagulation activity of RNA origami, aPTT (intrinsic coagulation pathway) and PT (extrinsic coagulation pathway) assays were used. As shown in FIGS. 1C-1H, results demonstrate that RNA origami anticoagulant has activity in both intrinsic and extrinsic pathway in human, porcine, and murine plasma.

### Example 2

Optimization of single-stranded DNA antidote. Single-molecule DNA antidotes containing complementary sequences of exosite-1 and exosite-2 binding RNA aptamers were designed. Varying the length between each antidote (from no spacer to 31 nucleotide-spacer) and reorganizing the position of antidotes, eight designs of single-molecule DNA antidotes were created. To optimize reversal activity, the single-molecule DNA antidotes were tested using aPTT assay (FIG. 2A). Anti-HEX21 without spacer has the highest reversal activity compared with other DNA antidote designs and better than mixed DNA antidotes. Surprisingly, Anti-HEX12 with 31 nucleotide-spacer enhanced anticoagulation activity. Experiments were also conducted to test the dose-dependent activity of DNA antidote (Anti-HEX21 without linker), as shown in FIG. 2B. The result demonstrated that this single-molecule DNA antidote provides dose-dependent response that would benefit for medical uses.

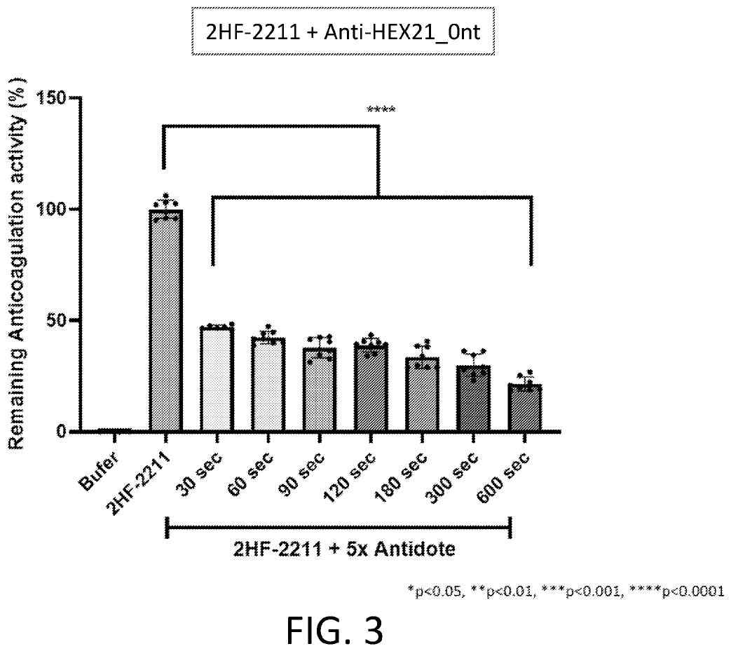

The rapid onset of action is crucial for specific reversal agent for anticoagulant. Therefore, experiments were conducted to test the reversal action of Anti-HEX21 without spacer using aPTT to evaluate clotting time in human plasma. As shown in FIG. 3, results demonstrated that the reversal action of single-molecule DNA antidote is as fast as 30 seconds and showed significant reduction of anticoagulation activity of RNA origami anticoagulant.

### Example 3

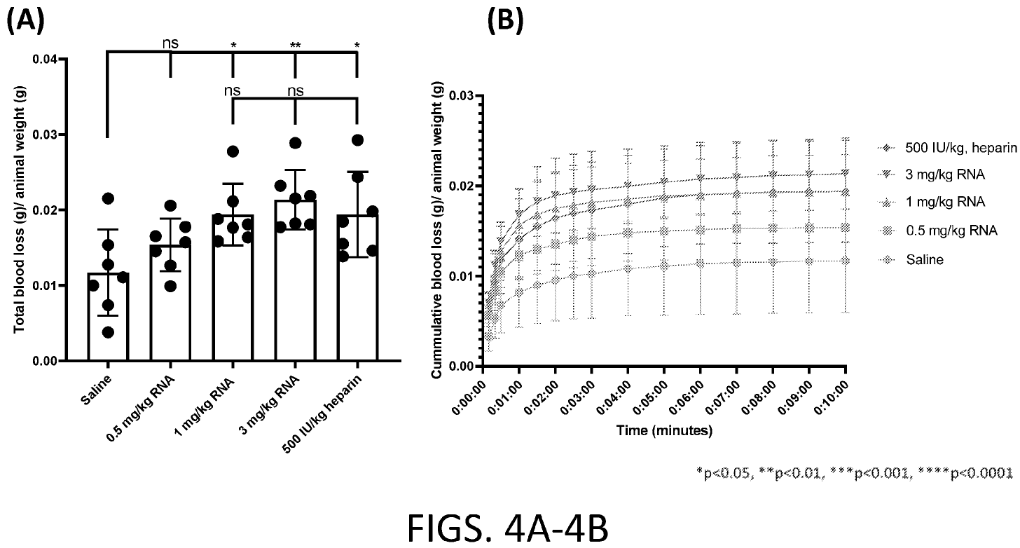

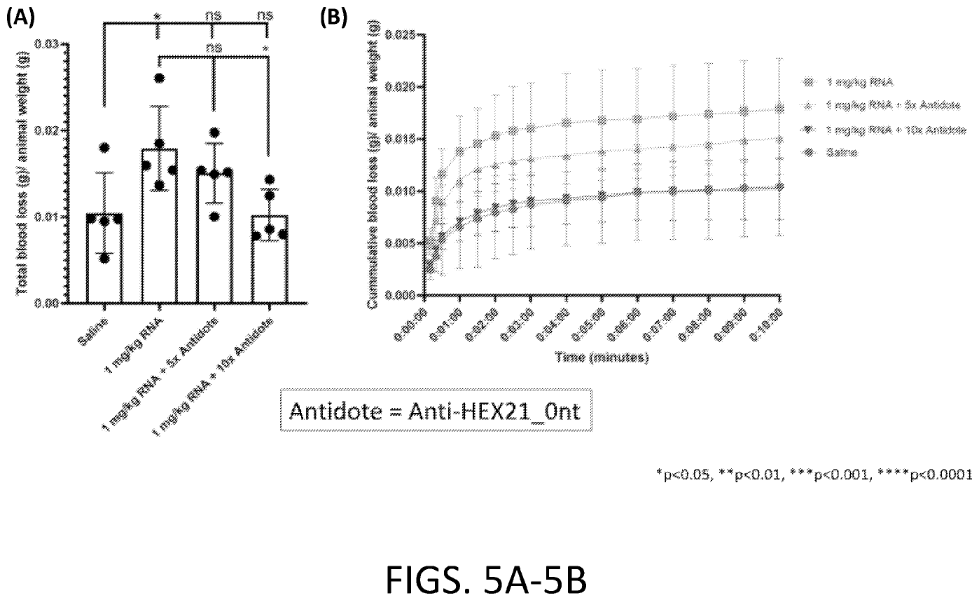

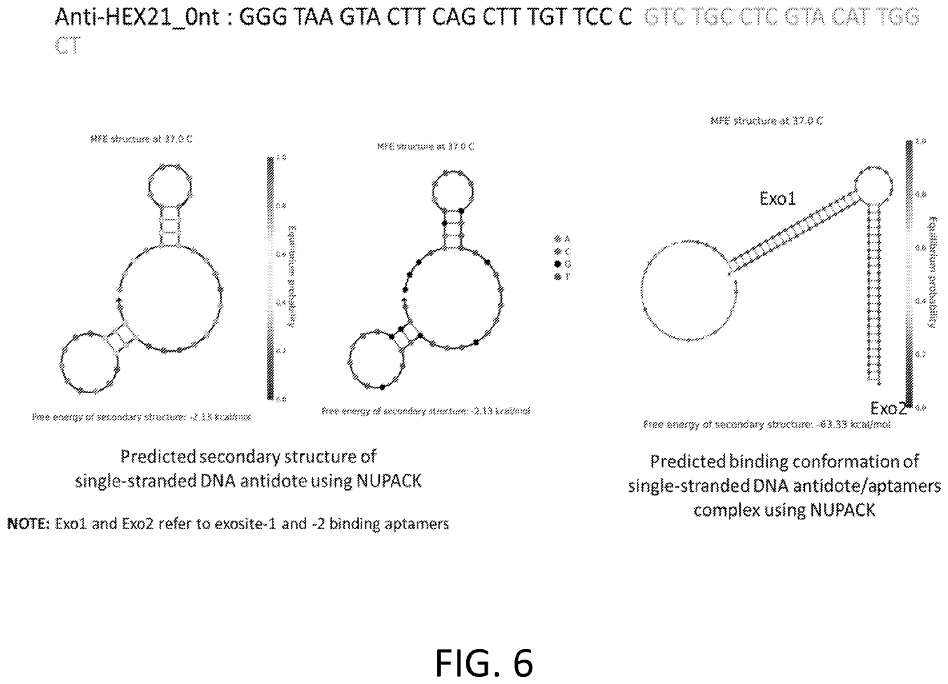

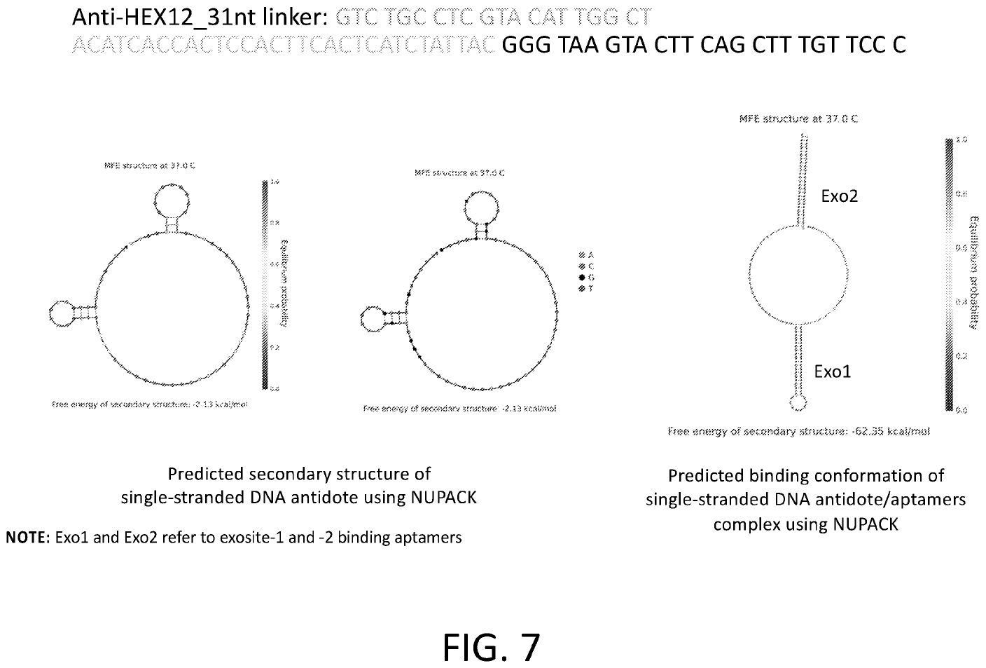

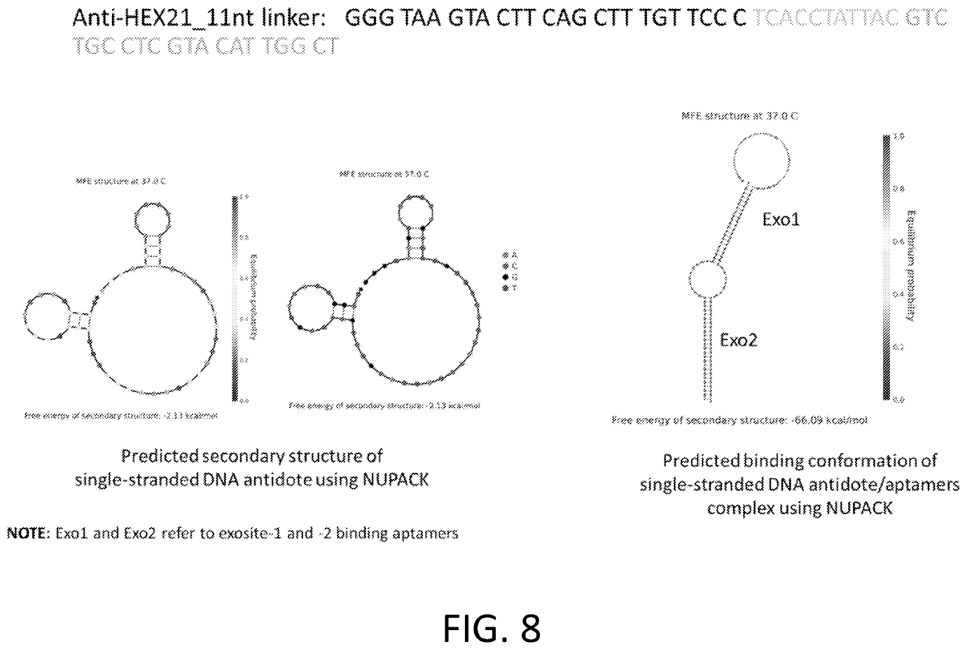

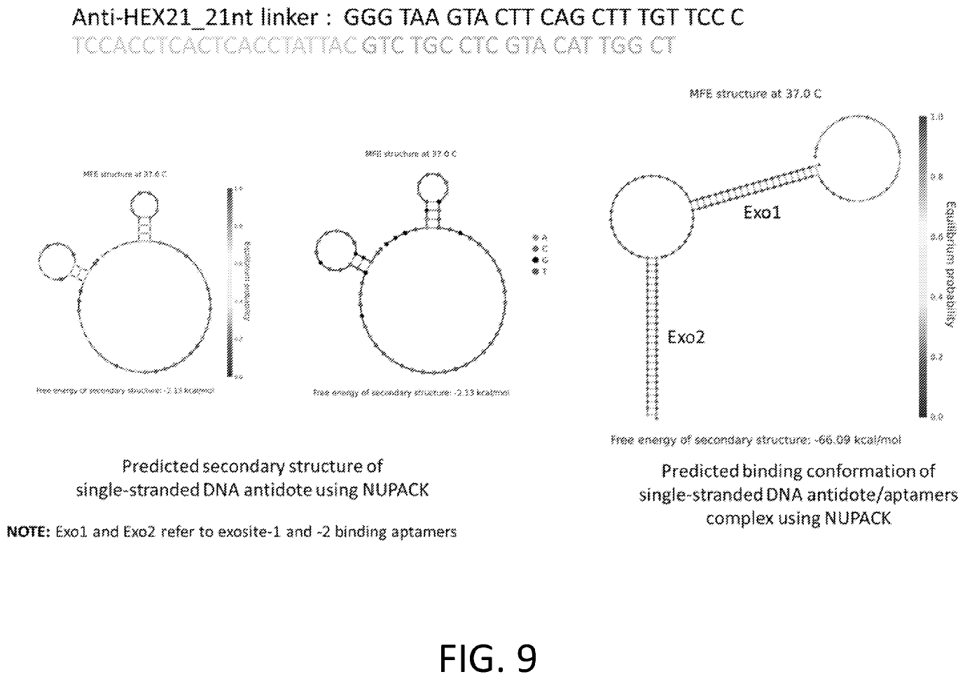

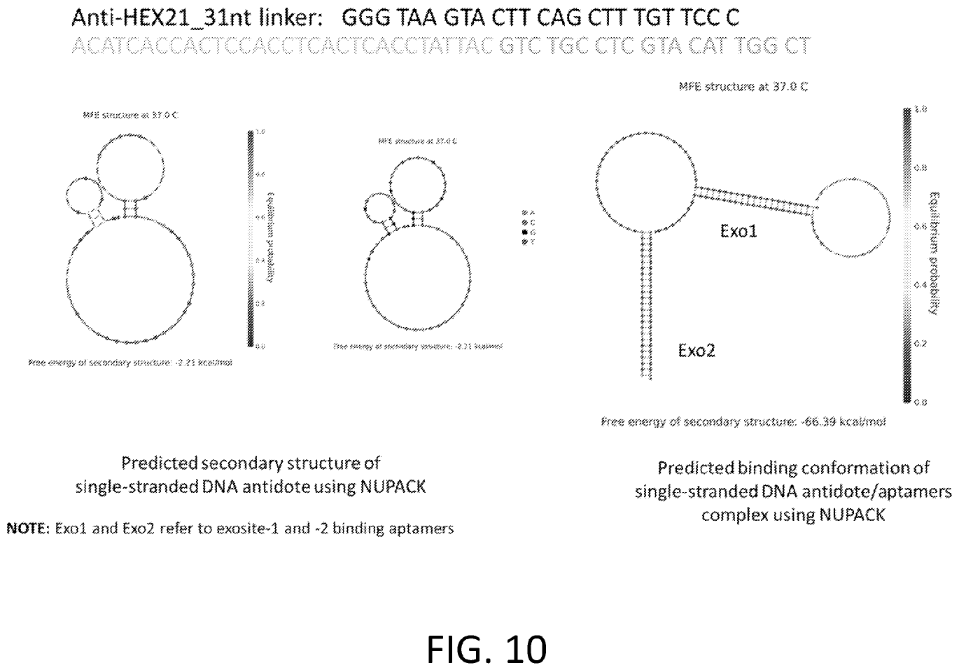

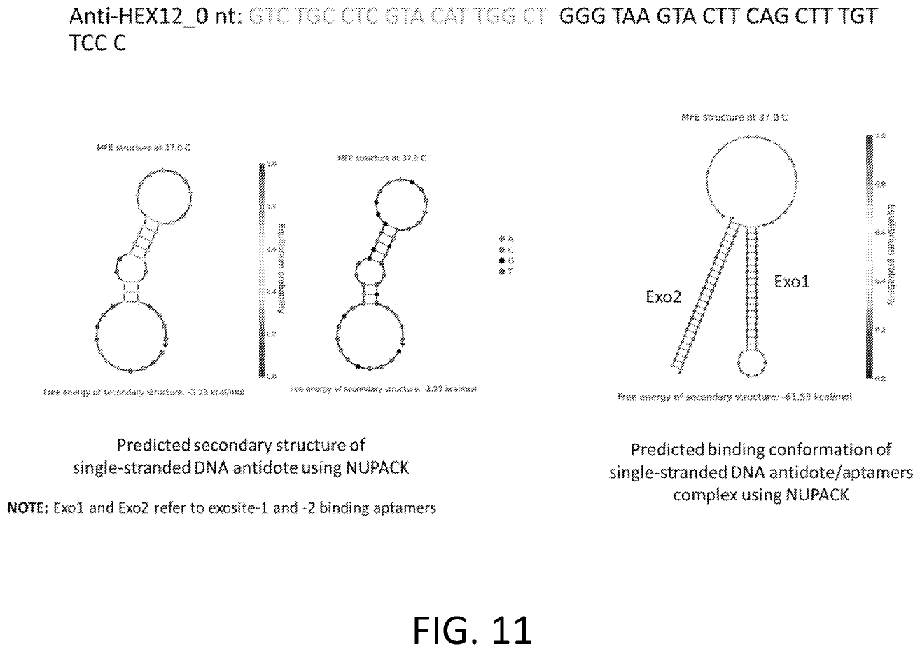

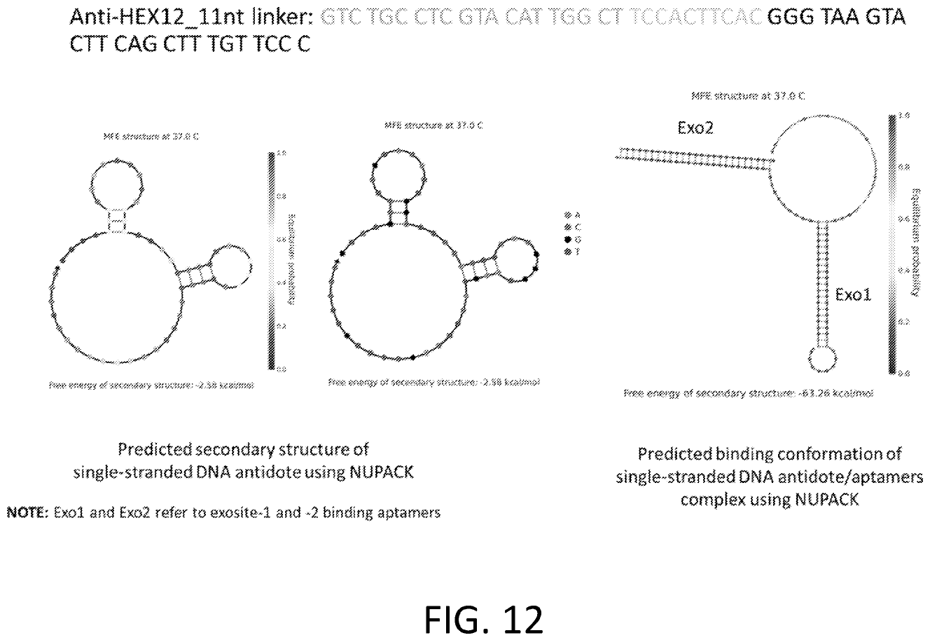

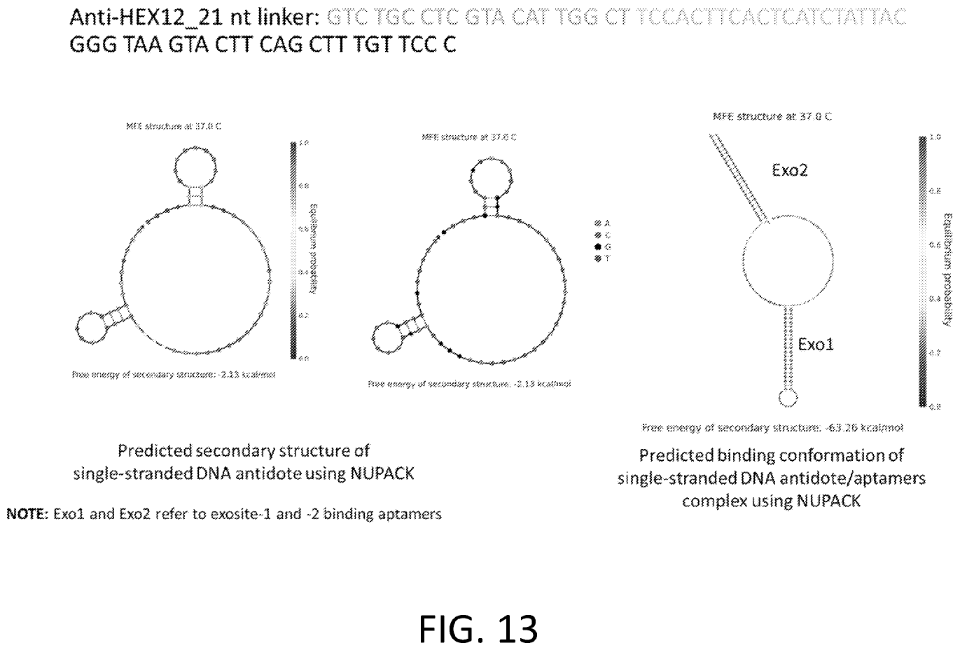

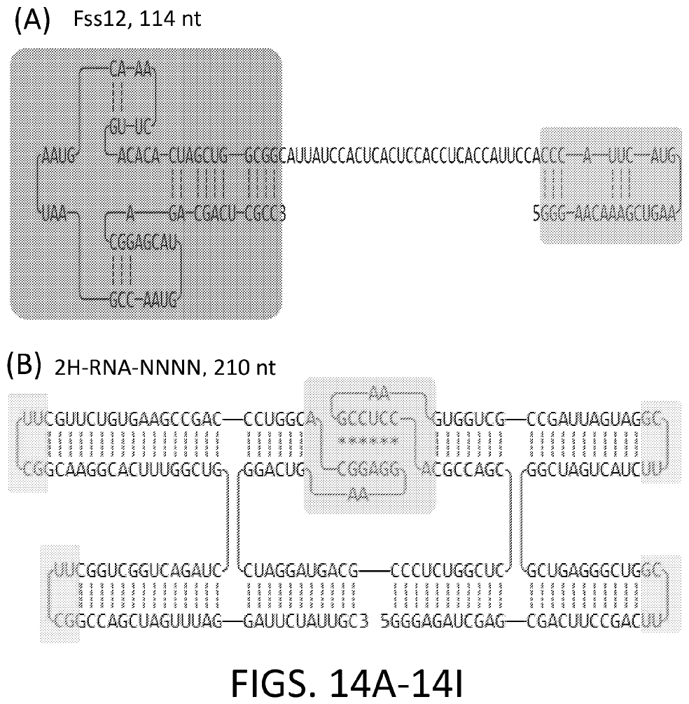

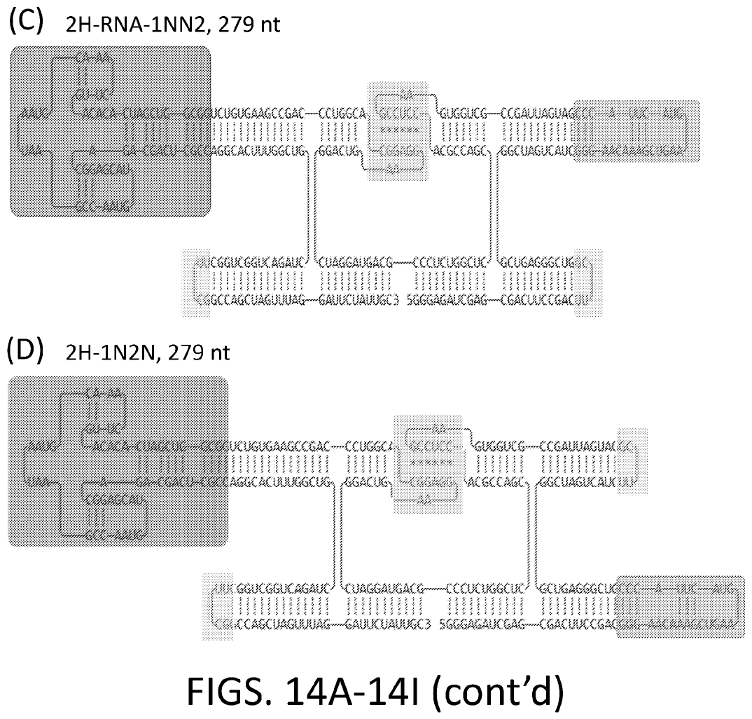

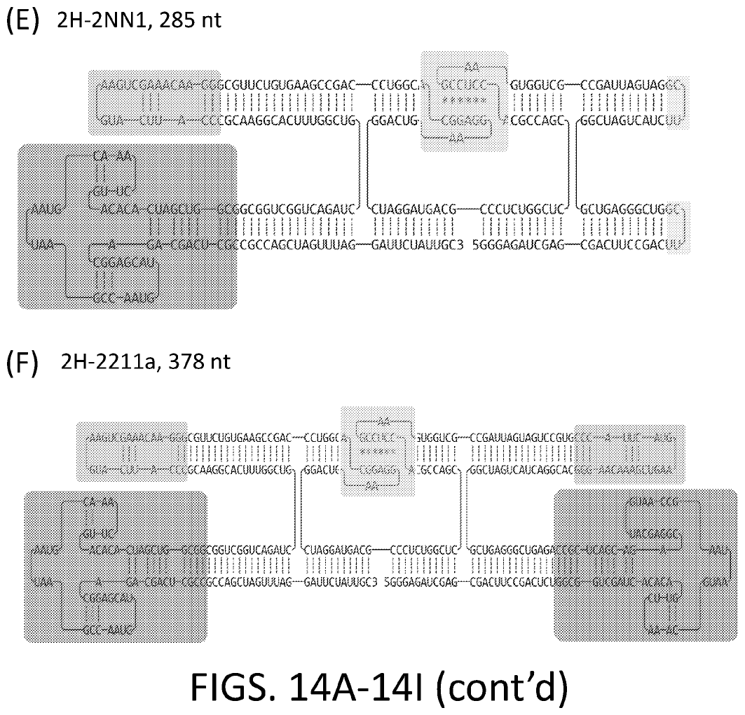

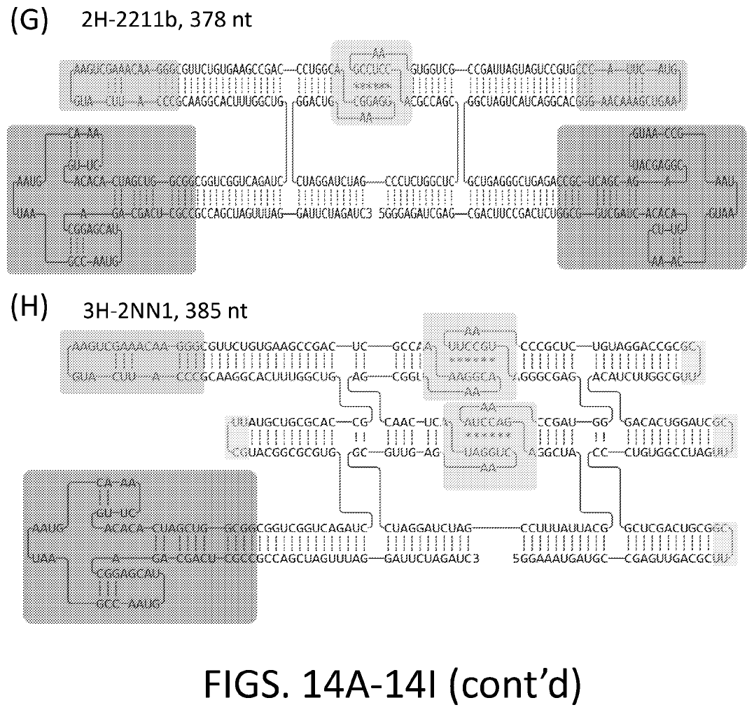

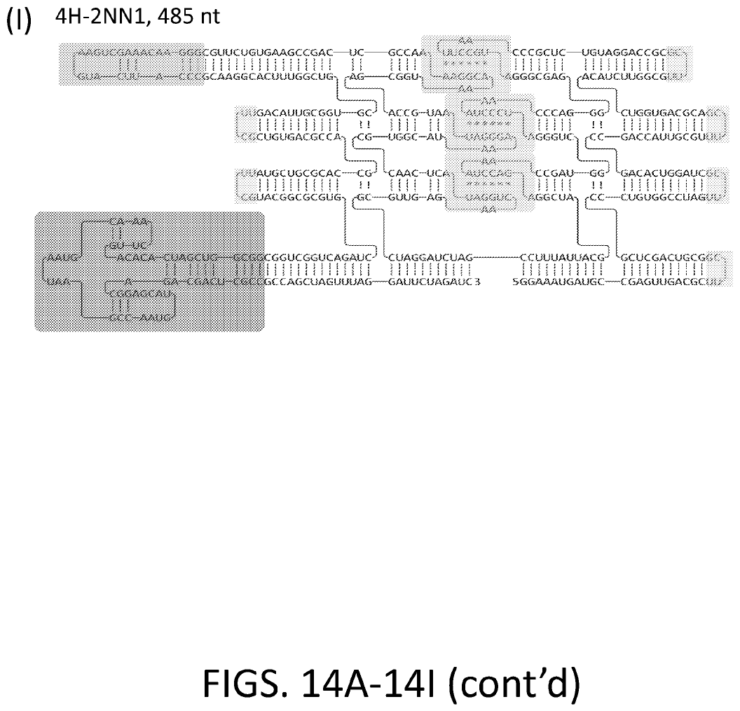

In vivo efficacy of RNA origami. Heparin is a traditional, intravenous anticoagulant that bas reversal agent, protamine. However, there are several drawbacks of heparin and protamine including unpredictable dose response, heparin induced thrombocytopenia (HIT), and batch-to-batch variabilities. Therefore, a novel, direct-thrombin inhibitor anticoagulant system was developed based on RNA origami technology in conjunction with a specific, single-molecule DNA antidote. Effective anticoagulation activity of RNA origami and reversal agent activity of single-molecule DNA antidote in vitro was successfully demonstrated using aPTT assay (FIGS. 1-3). Furthermore, experiments were conducted to test their activities in a murine model with liver laceration injury models (FIGS. 4-5). Results indicated that the anticoagulation activity of RNA origami was does-dependent as shown in FIG. 4. Total blood losses in high concentration of heparin (500 U/kg) and 1 mg/kg of RNA origami were not significantly different. These results demonstrated that RNA origami anticoagulant has efficacy in murine model.

The activity of reversal agents (Anti-HEX21 without spacers) was also tested in a murine model using liver laceration injury model. The results in FIG. 5 demonstrated that the total blood losses in control models (saline) and murine model treated with 1 mg/kg of RNA origami anticoagulant and 10 equivalence of Anti-HEX21 without linker were not significantly different.

What is claimed is:

1. A single-stranded nucleic acid molecule comprising at least one RNA aptamer reversal element that is complementary to at least a portion of an RNA aptamer having anti-coagulation activity.

2. The nucleic acid molecule of claim 1, wherein binding of the at least one RNA aptamer reversal element to the at least one RNA aptamer having anti-coagulation activity counteracts the anti-coagulation activity.

3. The nucleic acid molecule of claim 2, wherein the binding of the at least one RNA aptamer reversal element to the at least one RNA aptamer having anti-coagulant activity counteracts the anti-coagulation activity by at least about 25%.

4. The nucleic acid molecule of claim 2, wherein the binding of the at least one RNA aptamer reversal element to the at least one RNA aptamer having anti-coagulant activity counteracts the anti-coagulation activity by at least about 50%.

5. The nucleic acid molecule of claim 2, wherein the binding of the at least one RNA aptamer reversal element to the at least one RNA aptamer having anti-coagulant activity counteracts the anti-coagulation activity by at least about 75%.

6. The nucleic acid molecule of any of claims 1 to 5, wherein the at least one RNA aptamer reversal element binds to the at least one RNA aptamer with at least about 50% complementarity.

7. The nucleic acid molecule of any of claims 1 to 5, wherein the at least one RNA aptamer reversal element binds to the at least one RNA aptamer with at least about 75% complementarity.

8. The nucleic acid molecule of any of claims 1 to 5, wherein the at least one RNA aptamer reversal element binds to the at least one RNA aptamer with at least about 95% complementarity.

9. The nucleic acid molecule of any of claims 1 to 8, wherein the nucleic acid comprises at least two RNA aptamer reversal elements that are complementary to at least a portion of a single RNA aptamer having anti-coagulation activity.

10. The nucleic acid molecule of any of claims 1 to 8, wherein the nucleic acid comprises at least two RNA aptamer reversal elements that are complementary to at least a portion of two different RNA aptamers having anti-coagulation activity.

11. The nucleic acid molecule of claim 9 or claim 10, wherein the at least two RNA aptamer reversal elements are separated by a linker region.

12. The nucleic acid molecule of claim 11, wherein the linker region comprises from about 1 to about 50 nucleotides.

13. The nucleic acid molecule of claim 9 or claim 10, wherein the at least two RNA aptamer reversal elements are continuous and not separated by a linker region.

14. The nucleic acid molecule of any of claims 1 to 13, wherein the anti-coagulation activity of the RNA aptamer comprises inhibition of one or more of Factor XIIa, Factor XIIIa, Factor XIa, Factor IXa, Factor Xa, and/or von Willebrand factor.

15. The nucleic acid molecule of any of claims 1 to 13, wherein the anti-coagulation activity of the RNA aptamer comprises thrombin inhibition.

16. The nucleic acid molecule of claim 15, wherein the RNA aptamer is capable of binding exosite 1 of thrombin.

17. The nucleic acid molecule of claim 15, wherein the RNA aptamer is capable of binding exosite 2 of thrombin.

18. The nucleic acid molecule of claim 15, wherein the RNA aptamer is an anti-thrombin RNA_{R9D-14T} aptamer or a derivative thereof.

19. The nucleic acid molecule of claim 15, wherein the RNA aptamer comprises an anti-thrombin Toggle-25t RNA aptamer or a derivative thereof.

20. The nucleic acid molecule of any of claims 1 to 19, wherein the at least one RNA aptamer reversal element comprises a sequence that is at least 80% identical to SEQ ID NOs: 1 or 2.

21. The nucleic acid molecule of any of claims 1 to 20, wherein the nucleic acid comprises a sequence that is at least 80% identical to any of SEQ ID NOs: 3-10.

22. A vector comprising any of the nucleic acids of claim 21.

23. A therapeutic composition comprising:
any of the nucleic acid molecules of claims 1 to 21; and
a pharmaceutically acceptable excipient, solvent, carrier, or diluent.

24. A method of modulating coagulation in a subject in need thereof, the method comprising administering the composition of claim 23 to the subject.

25. The method of claim 25, wherein the administration of the composition of claim 23 to the subject is performed after the subject has been administered a composition comprising an RNA aptamer having anti-coagulation activity.

26. A system or kit for modulating coagulation, the system comprising:
any of the nucleic acid molecules of claims 1 to 21; and
an RNA aptamer having anti-coagulation activity.


SEQUENCE LISTING (29 sequences)

SEQ ID NO: 1 - DNA, 19 bp, synthetic construct
  source 1..19: mol_type = other DNA
gtctgcctcgtcattggct

SEQ ID NO: 2 - DNA, 25 bp, synthetic construct
  source 1..25: mol_type = other DNA
gggtaagtacttcagctttgttccc

SEQ ID NO: 3 - DNA, 45 bp, synthetic construct
  source 1..45: mol_type = other DNA
gggtaagtacttcagctttgttcccgtctgcctcgtacattggct

SEQ ID NO: 4 - DNA, 56 bp, synthetic construct
  source 1..56: mol_type = other DNA
gggtaagtacttcagctttgttccctcacctattacgtctgcctcgtacattggct

SEQ ID NO: 5 - DNA, 66 bp, synthetic construct
  source 1..66: mol_type = other DNA
gggtaagtacttcagctttgttccctccacctcactcacctattacgtctgcctcgtacattggct

SEQ ID NO: 6 - DNA, 76 bp, synthetic construct
  source 1..76: mol_type = other DNA
gggtaagtacttcagctttgttcccacatcaccactccacctcactcacctattacgtctgcctcgtacattggct

SEQ ID NO: 7 - DNA, 45 bp, synthetic construct
  source 1..45: mol_type = other DNA
gtctgcctcgtacattggctgggtaagtacttcagctttgttccc

SEQ ID NO: 8 - DNA, 55 bp, synthetic construct
  source 1..55: mol_type = other DNA
gtctgcctcgtacattggcttccacttcacgggtaagtacttcagctttgttccc

SEQ ID NO: 9 - DNA, 66 bp, synthetic construct
  source 1..66: mol_type = other DNA
gtctgcctcgtacattggcttccacttcactcatctattacgggtaagtacttcagctttgttccc

SEQ ID NO: 10 - DNA, 76 bp, synthetic construct
  source 1..76: mol_type = other DNA
gtctgcctcgtacattggctacatcaccactccacttcactcatctattacgggtaagtacttcagctttgttccc

SEQ ID NO: 11 - RNA, 210 nt, synthetic construct
  source 1..210: mol_type = other RNA
gggagatcgagcgacttccgacttcggtcgggagtcgggctagtcatcttcggatgattagccgctggtgaagcctccacgccagcctcggtctcccgcagtaggatcggactgaaggaggcacggtcccagccgaagtgtcttgcttcggcaaggcactttggctgctagactggctggcttcggccagctagtttaggattctattgc

SEQ ID NO: 12 - RNA, 279 nt, synthetic construct
  source 1..279: mol_type = other RNA
gggagatcgagcgacttccgacttcggtcgggagtcgggctagtcatcgggaacaaagctgaagtacttacccgatgattagccgctggtgaagcctccacgccagcctcggtctcccgcagtaggatcggactgaaggaggcacggtcccagccgaagtgtctggcggtcgatcacacagttcaaacgtaataagccaatgtacgaggcagacgactcgccaggcactttggctgctagactggctggcttcggccagctagtttaggattctattgc

SEQ ID NO: 13 - RNA, 279 nt, synthetic construct
  source 1..279: mol_type = other RNA
gggagatcgagcgacttccgacgggaacaaagctgaagtacttacccgtcgggagtcgggctagtcatcttcggatgattagccgctggtgaagcctccacgccagcctcggtctcccgcagtaggatcggactgaaggaggcacggtcccagccgaagtgtctggcggtcgatcacacagttcaaacgtaataagccaatgtacgaggcagacgactcgccaggcactttggctgctagactggctggcttcggccagctagtttaggattctattgc

SEQ ID NO: 14 - RNA, 285 nt, synthetic construct
  source 1..285: mol_type = other RNA
gggagatcgagcgacttccgacttcggtcgggagtcgggctagtcatcttcggatgattagccgctggtgaagcctccacgccagcctcggtctcccgcagtaggatcggactgaaggaggcacggtcccagccgaagtgtcttgcgggaacaaagctgaagtacttacccgcaaggcactttggctgctagactggctggcggcggtcgatcacacagttcaaacgtaataagccaatgtacgaggcagacgactcgccgccagctagtttaggattctattgc

SEQ ID NO: 15 - RNA, 114 nt, synthetic construct
  source 1..114: mol_type = other RNA
gggaacaaagctgaagtacttacccaccttaccactccacctcactcacctattacggcggtcgatcacacagttcaaacgtaataagccaatgtacgaggcagacgactcgcc

SEQ ID NO: 16 - RNA, 378 nt, synthetic construct
  source 1..378: mol_type = other RNA
gggagatcgagcgacttccgactctggcggtcgatcacacagttcaaacgtaataagccaatgtacgaggcagacgactcgccagagtcgggagtcgggctagtcatcaggcacgggaacaaagctgaagtacttacccgtgcctgatgattagccgctggtgaagcctccacgccagcctcggtctcccgcagtaggatcggactgaaggaggcacggtcccagccgaagtgtcttgcgggaacaaagctgaagtacttacccgcaaggcactttggctgctagactggctggcggcggtcgatcacacagttcaaacgtaataagccaatgtacgaggcagacgactcgccgccagctagtttaggattctattgc

SEQ ID NO: 17 - RNA, 378 nt, synthetic construct
  source 1..378: mol_type = other RNA
gggagatcgagcgacttccgactctggcggtcgatcacacagttcaaacgtaataagccaatgtacgaggcagacgactcgccagagtcgggagtcgggctagtcatcaggcacgggaacaaagctgaagtacttacccgtgcctgatgattagccgctggtgaagcctccacgccagcctcggtctcccgatctaggatcggactgaaggaggcacggtcccagccgaagtgtcttgcgggaacaaagctgaagtacttacccgcaaggcactttggctgctagactggctggcggcggtcgatcacacagttcaaacgtaataagccaatgtacgaggcagacgactcgccgccagctagtttaggattctagatc

SEQ ID NO: 18 - RNA, 385 nt, synthetic construct
  source 1..385: mol_type = other RNA
ggaaatgatgccgagttgacgcttcggcgtcagctcgccctgtggcctagttcgctaggtcacagacatcttggcgttcgcgccaggatgtctcgcccaattccgtagggcgagggtagccaaatccagaggctagcattatttccgatctaggatcgcgttgagaactggatactcaacagcggtaaacggaaaaccgctcagccgaagtgtcttgcgggaacaaagctgaagtacttacccgcaaggcactttggctggccacgcgtcgtattcgtacggcgcgtgctagactggctggcggcggtcgatcacacagttcaaacgtaataagccaatgtacgaggcagacgactcgccgccagctagtttaggattctagatc

SEQ ID NO: 19 - RNA, 485 nt, synthetic construct
  source 1..485: mol_type = other RNA
ggaaatgatgccgagttgacgcttcggcgtcagctcgccctgtggcctagttcgctaggtcacagccgaccattgcgtttcgacgcagtggtcacatcttggcgttcgcgccaggatgtctcgcccaattccgtagggcgaggggacccaaatccctagggtcggtagccaaatccagaggctagcattatttccgatctaggatcgcgttgagaactggatactcaaccgtggcataaagggataatgccaagcggtaaacggaaaaccgctcagccgaagtgtcttgcgggaacaaagctgaagtacttacccgcaaggcactttggctgcgtggcgttacagttcgctgtgacgccagccacgcgtcgtattcgtacggcgcgtgctagactggctggcggcggtcgatcacacagttcaaacgtaataagccaatgtacgaggcagacgactcgccgccagctagtttaggattctagatc

SEQ ID NO: 20 - DNA, 261 bp, synthetic construct
  source 1..261: mol_type = other DNA
cactttcagccctcttatcctcggcggatccttctaatacgactcactatagggagatcgagcgacttccgacttcggtcgggagtcgggctagtcatcttcggatgattagccgctggtgaagcctccacgccagcctcggtctcccgcagtaggatcggactgaaggaggcacggtcccagccgaagtgtcttgcttcggcaaggcactttggctgctagactggctggcttcggccagctagtttaggattctattgc

SEQ ID NO: 21 - DNA, 330 bp, synthetic construct
  source 1..330: mol_type = other DNA
cactttcagccctcttatcctcggcggatccttctaatacgactcactatagggagatcgagcgacttccgacttcggtcgggagtcgggctagtcatcgggaacaaagctgaagtacttacccgatgattagccgctggtgaagcctccacgccagcctcggtctcccgcagtaggatcggactgaaggaggcacggtcccagccgaagtgtctggcggtcgatcacacagttcaaacgtaataagccaatgtacgaggcagacgactcgccaggcactttggctgctagactggctggcttcggccagctagtttaggattctattgc

SEQ ID NO: 22 - DNA, 330 bp, synthetic construct
  source 1..330: mol_type = other DNA
cactttcagccctcttatcctcggcggatccttctaatacgactcactatagggagatcgagcgacttccgacgggaacaaagctgaagtacttacccgtcgggagtcgggctagtcatcttcggatgattagccgctggtgaagcctccacgccagcctcggtctcccgcagtaggatcggactgaaggaggcacggtcccagccgaagtgtctggcggtcgatcacacagttcaaacgtaataagccaatgtacgaggcagacgactcgccaggcactttggctgctagactggctggcttcggccagctagtttaggattctattgc

SEQ ID NO: 23 - DNA, 336 bp, synthetic construct
  source 1..336: mol_type = other DNA
cactttcagccctcttatcctcggcggatccttctaatacgactcactatagggagatcgagcgacttccgacttcggtcgggagtcgggctagtcatcttcggatgattagccgctggtgaagcctccacgccagcctcggtctcccgcagtaggatcggactgaaggaggcacggtcccagccgaagtgtcttgcgggaacaaagctgaagtacttacccgcaaggcactttggctgctagactggctggcggcggtcgatcacacagttcaaacgtaataagccaatgtacgaggcagacgactcgccgccagctagtttaggattctattgc

SEQ ID NO: 24 - DNA, 165 bp, synthetic construct
  source 1..165: mol_type = other DNA
cactttcagccctcttatcctcggcggatccttctaatacgactcactatagggaacaaagctgaagtacttacccaccttaccactccacctcactcacctattacggcggtcgatcacacagttcaaacgtaataagccaatgtacgaggcagacgactcgcc

SEQ ID NO: 25 - DNA, 429 bp, synthetic construct
  source 1..429: mol_type = other DNA
cactttcagccctcttatcctcggcggatccttctaatacgactcactatagggagatcgagcgacttccgactctggcggtcgatcacacagttcaaacgtaataagccaatgtacgaggcagacgactcgccagagtcgggagtcgggctagtcatcaggcacgggaacaaagctgaagtacttacccgtgcctgatgattagccgctggtgaagcctccacgccagcctcggtctcccgcagtaggatcggactgaaggaggcacggtcccagccgaagtgtcttgcgggaacaaagctgaagtacttacccgcaaggcactttggctgctagactggctggcggcggtcgatcacacagttcaaacgtaataagccaatgtacgaggcagacgactcgccgccagctagtttaggattctattgc

SEQ ID NO: 26 - DNA, 429 bp, synthetic construct
  source 1..429: mol_type = other DNA
cactttcagccctcttatcctcggcggatccttctaatacgactcactatagggagatcgagcgacttccgactctggcggtcgatcacacagttcaaacgtaataagccaatgtacgaggcagacgactcgccagagtcgggagtcgggctagtcatcaggcacgggaacaaagctgaagtacttacccgtgcctgatgattagccgctggtgaagcctccacgccagcctcggtctcccgatctaggatcggactgaaggaggcacggtcccagccgaagtgtcttgcgggaacaaagctgaagtacttacccgcaaggcactttggctgctagactggctggcggcggtcgatcacacagttcaaacgtaataagccaatgtacgaggcagacgactcgccgccagctagtttaggattctagatc

SEQ ID NO: 27 - DNA, 472 bp, synthetic construct
  source 1..472: mol_type = other DNA
cggccagtgaattcgagctcggtacccgggagatctcactttcagccctcttatcctcggccgatccttctaatacgactcactataggaaatgatgccgagttgacgcttcggcgtcagctcgccctgtggcctagttcgctaggtcacagacatcttggcgttcgcgccaggatgtctcgcccaattccgtagggcgagggtagccaaatccagaggctagcattatttccgatctaggatcgcgttgagaactggatactcaacagcggtaaacggaaaaccgctcagccgaagtgtcttgcgggaacaaagctgaagtacttacccgcaaggcactttggctggccacgcgtcgtattcgtacggcgcgtgctagactggctggcggcggtcgatcacacagttcaaacgtaataagccaatgtacgaggcagacgactcgccgccagctagtttaggattctagatc

SEQ ID NO: 28 - DNA, 572 bp, synthetic construct
  source 1..572: mol_type = other DNA
cggccagtgaattcgagctcggtacccgggagatctcactttcagccctcttatcctcggccgatccttctaatacgactcactataggaaatgatgccgagttgacgcttcggcgtcagctcgccctgtggcctagttcgctaggtcacagccgaccattgcgtttcgacgcagtggtcacatcttggcgttcgcgccaggatgtctcgcccaattccgtagggcgaggggacccaaatccctagggtcggtagccaaatccagaggctagcattatttccgatctaggatcgcgttgagaactggatactcaaccgtggcataaagggataatgccaagcggtaaacggaaaaccgctcagccgaagtgtcttgcgggaacaaagctgaagtacttacccgcaaggcactttggctgcgtggcgttacagttcgctgtgacgccagccacgcgtcgtattcgtacggcgcgtgctagactggctggcggcggtcgatcacacagttcaaacgtaataagccaatgtacgaggcagacgactcgccgccagctagtttaggattctagatc

SEQ ID NO: 29 - DNA, 3118 bp, synthetic construct
  source 1..3118: mol_type = other DNA
catcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggagatctcactttcagccctcttatcctcggccgatccttctaatacgactcactatagggagatcgagcgacttccgactctggcggtcgatcacacagttcaaacgtaataagccaatgtacgaggcagacgactcgccagagtcgggagtcgggctagtcatcaggcacgggaacaaagctgaagtacttacccgtgcctgatgattagccgctggtgaagcctccacgccagcctcggtctcccgatctaggatcggactgaaggaggcacggtcccagccgaagtgtcttgcgggaacaaagctgaagtacttacccgcaaggcactttggctgctagactggctggcggcggtcgatcacacagttcaaacgtaataagccaatgtacgaggcagacgactcgccgccagctagtttaggattctagatctctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccg